SEQUENCE LISTING (7 sequences)

SEQ ID NO: 1 - protein, 330 aa, Mycobacterium tuberculosis
MISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 2 - protein, 330 aa, synthetic construct
  REGION 1..330: note = synthetic
MISEFPELNTARGPIDTADLGVTLMHEHVFIMTTEIAQNYPEAWGDEDKRVAGAIARLVELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 3 - protein, 330 aa, synthetic construct
  REGION 1..330: note = synthetic
MISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAYKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 4 - protein, 716 aa, synthetic construct
  REGION 1..716: note = synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 5 - protein, 716 aa, synthetic construct
  REGION 1..716: note = synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFPELNTARGPIDTADLGVTLMHEHVFIMTTEIAQNYPEAWGDEDKRVAGAIARLVELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAHKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 6 - protein, 716 aa, synthetic construct
  REGION 1..716: note = synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFPELNTARGPIDTADLGVTLMHEHVFIMTTEIAQNYPEAWGDEDKRVAGAIARLGELKARGVDTIVDLTVIGLGRYIPRIARVAAATELNIVVATGLYTYNDVPFYFHYLGPGAQLDGPEIMTDMFVRDIEHGIADTGIKAGILKCATDEPGLTPGVERVLRAVAQAYKRTGAPISTHTHAGLRRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNWHYLHIHNDVIPALKQHGVTDEQLHTMLVDNPRRIFERQGGYQ

SEQ ID NO: 7 - protein, 209 aa, Erwinia amylovora
MNYHIIPVTAFAQNCSVIWCPETGEAALVDPGGDAQKIKHEIAALGLTVKQILLTHGHLDHVGAAAELAEHYDVPIVGPHKADKFWLDGLPQQSQMFGFDECAPLTPDRWLQEGEVVLVGNTVLEVLLCPGHTPGHIVFFERKARLLVSGDVIFSGGVGRSDFPQGSHQDLIASILNKLLPLGDDVTFIAGHGPMSTLGRERISNPFLQ

(19) United States
(12) Patent Application Publication
(10) Pub. No.: US 2023/0416704 A1
(43) Pub. Date: Dec. 28, 2023

(54) LACTONASE AND STABILIZED MUTANTS THEREOF FOR TREATING FUNGAL INFECTIONS IN PLANTS

(71) Applicant: MIGAL GALILEE RESEARCH INSTITUTE LTD., Kiryat Shmona (IL)

(72) Inventors: Livnat AFRIAT-JURNOU, Kfar-Szold (IL); Dov PRUSKY, Tel Aviv (IL)

(21) Appl. No.: 18/336,378

(22) Filed: Jun. 16, 2023

Related U.S. Application Data
(63) Continuation-in-part of application No. PCT/IL2021/051485, filed on Dec. 14, 2021.
(60) Provisional application No. 63/126,277, filed on Dec. 16, 2020.

(51) Int. Cl. C12N 9/16; A01P 3/00; A01N 63/50

(52) U.S. Cl. CPC C12N 9/16; C12Y 301/08001; A01P 3/00; A01N 63/50

(57) ABSTRACT

Methods for treating or preventing infection of a fungus secreting patulin in plants or products made therefrom; and for reducing the concentration of patulin in plants, products made therefrom, or non-plant food products, using a lactonase such as an acyl-homoserine lactonase, e.g., a phosphotriesterase-like lactonase; or the wild-type putative parathion hydrolase from M. tuberclorosis (PPH) or a mutant thereof, or a functional fragment thereof.

### CROSS-REFERENCE TO RELATED APPLICATIONS

The present application is a continuation-in-part of International Application No. PCT/IL2021/051485, filed Dec. 14, 2021, designating the U.S. and published as WO 2022/130378 on Jun. 23, 2022, which claims the benefit of U.S. Provisional Patent Application No. 63/126,277, filed Dec. 16, 2020. Any and all applications for which a foreign or domestic priority claim is identified above and/or in the Application Data Sheet as filed with the present application are hereby incorporated by reference under 37 CFR 1.57.

### SEQUENCE LISTING IN ELECTRONIC FORMAT

The present application is being filed along with an Electronic Sequence Listing in ST.26 format. The Electronic Sequence Listing is provided as a file entitled BEN046_003P1_SL.xml created and last saved on Jun. 15, 2023, which is approximately 12.1 KB in size. The information in the Electronic Sequence Listing is incorporated herein by reference in its entirety in accordance with 35 U.S.C. § 1.52(e).

### FIELD

The present invention provides methods for treating or preventing infection of a fungus secreting patulin in plants or products made therefrom; and for reducing the concentration of patulin in plants, products made therefrom, or non-plant food products.

### BACKGROUND

Microorganisms associated with the fruit microbiome are found on the surfaces (epiphytes) or in the tissues of the fruit (endophytes). The recent knowledge gained from microbial community analysis indicates location dependence and is relevant to biological control to prevent post-harvest fruit pathology (Abdelfattah et al., 2021). The demand to study the epiphytic microbiome is increasing in light of the understanding that raw-eaten plants seem to be a source for microbes that are a part of the gut microbiome and a source for pathogens that might play a role in human health (Berg et al., 2017). Among other microbes, filamentous fungi are found in raw food, and most of them produce metabolites that are of risk to human health (Walsh et al., 2004; Luciano-Rosario et al., 2020). Some of them are also associated with human infections (Walsh et al., 2004). For example, the plant's pathogenic species P. citrinum, chlysogenunt, P. digitaturn, P. marneffei, and P. expansum can cause human infection through inhalation and sometimes ingestion, causing necrotizing esophagitis, endophthalmitis, keratitis, and asthma (Walsh et al., 2004).

Penicillium expansum is a necrotrophic wound fungal pathogen that secrets various virulence factors to kill host cells, including cell wall degrading enzymes (CWDEs), proteases, and also produces mycotoxins such as patulin (Luciano-Rosario et al., 2020). During the interaction between P. expansum and its fruit host, these virulence factors are strictly modulated by intrinsic regulators and extrinsic environmental factors (Luciano-Rosario et al., 2020; Barad et al., 2012; Kumar et al., 2018a). P. expansum also has a cytotoxic effect that can lead to health risks in agriculture workers (Madsen et al., 2020). In recent years, there has been a rapid increase in the research towards understanding the molecular mechanisms, including the involvement of mycotoxins in pathogenicity of P. expansum, especially after sequencing of the genomes of P. expansum and closely related Penicillium species (Ianiri et al., 2013). Patulin is a lactone-based mycotoxin produced by P. expansum, most commonly found in colonized apples. The amount of patulin in apple products is generally viewed as a measure of apple quality. Due to the high toxicity of patulin, many toxicological regulatory organizations worldwide have set a maximum limit for patulin levels in foods, and studying the genes and enzymes involved in its biological degradation are of great interest (Ianiri et al., 2013).

Quorum sensing (QS) is one of the most studied regulatory mechanisms that enable bacteria to monitor their population density, integrate intercellular signals, and coordinate gene expression to benefit the bacterial community in various environments (Waters and Bassler, 2005; Fuqua et al., 1996). By sensing the extracellular concentration of secreted auto-inducer molecules, quorum-sensing signaling molecules (QSMs), the expression of various genes, such as genes involved in biofilm formation, antibiotics production, and virulence factors are affected (Aframian and Eldar, 2020). For example, N-acyl homoserine lactones (AHLs) are the most common auto-inducers of Gram-negative bacteria (Poonguzhali et al., 2007). Many bacterial pathogens utilize AHLs to coordinate pathogenicity (Uroz et al., 2009), including Pantoea stewartii, Erwinia carotovora, Pseudomonas syringae, and Xanthomonas campestris (Von Bodman et al., 2003). QS systems are appealing antimicrobial therapeutic targets, mainly since they regulate virulence gene expression in bacterial pathogens (Remy et al., 2018). Targeting QS will attenuate the production of virulence factors without exerting selective pressure and potentially lower the chances of resistance development. Strategies that target QS are named quorum-quenching strategies. Interestingly, patulin can act as a QS inhibitor molecule; for example, in Pseudomonas aeruginosa it downregulated QS-regulated genes (Rasmussen et al., 2005). Patulin also inhibited QS-regulated biofilm formation in Methylobacterium oryzae (a Gram-negative bacteria) and affected bacterial cell numbers (Afonso et al., 2020). Co-growth of Methylobacterium oryzae and P. expansum spores induced a differential gene expression of genes involved in patulin biosynthetic pathway clusters (such as the gene coding for isoepoxydon dehydrogenase) (Afonso et al., 2021). Therefore, patulin production may play a role in inter-kingdom communication.

Several enzymes which degrade bacterial AHLs were characterized, such as acylases (Lin et al., 2003) and lactonases (Afriat et al., 2006; Liu et al., 2007; Zhang et al., 2019). AHL lactonases proficiently hydrolyze the lactone ring in AHLs, leading to inhibition of QS related functions such as biofilm, virulence factors, and infections (Remy et al., 2016). Filamentous fungi also possess AHL lactonase activity. Intracellular AHL lactonases were identified in Coprinopsis cinerea and characterized, these fungal lactonases belong to the metallo-β-lactamase family (MBL, PF00753) exhibited similar AHL hydrolyzing activity as AiiA from Bacillus thuringiensis (Hornby et al., 2001).

Fungi and bacteria co-exist in various habitats, and are thought to be engaged in inter-kingdom communications such as QSM by cross detection or degradation (Rodrigues and C̆ernáková, 2020; Wongsuk et al., 2016). QS molecules that play a role in fungal pathogenicity were studied in yeasts and filamentous fungi, such as Candida albicans, Candida dubliniensis, Aspergillus niger, Aspergillus nidulans, and Fusarium graminearum (Wongsuk et al., 2016; Venkatesh and Keller, 2019).

The apple microbiome depends on many factors such as genotype and management practices (Abdelfattah et al., 2021; Angeli et al., 2019; Cui et al., 2021). A recent study indicated that the abundance and distribution of bacterial phyla in the “Royal Gala” apple fruit were consistent in most examined countries (Abdelfattah et al., 2021). The most abundant bacterial genera were Sphingomonas, Erwinia, Pseudomonas, Bacillus, unidentified Oxalobacteraceae, Methylobacterium, and unidentified Microbacteriaceae (Abdelfattah et al., 2021). In terms of the apple fungi community, in all countries, the most dominant phyla were Ascomycota (79.8%) then Basidiomycota (9.3%) (Abdelfattah et al., 2021). Two of the major microbial pathogens that affect apple production are the fungi P. expansum, causing the post-harvest disease blue mold (Luciano-Rosario et al., 2020) from the Ascomycota phyla, and the Gram-negative bacterial phytopathogen Erwinia amylovora from Erwinia, the cause of fire-blight disease. However, a deep understanding of the molecular mechanisms involving the epiphytic microbial population's interaction is still needed (Abdelfattah et al., 2021).

### SUMMARY

In one aspect, the present invention provides a method for treating or preventing infection of a fungus in a plant or a part, organ or a propagation material thereof, or in a product made from said plant, part, organ or propagation material, said fungus secreting patulin, and said method comprising applying a lactonase or a functional fragment thereof on said plant, part, organ or propagation material; or to said product.

In another aspect, the present invention provides a method for reducing the concentration of patulin in a plant or a part, organ or a propagation material thereof; in a product made from said plant, part, organ or propagation material; or in a non-plant food product, said method comprising applying a lactonase or a functional fragment thereof on said plant, part, organ or propagation material; or to said product.

### BRIEF DESCRIPTION OF THE DRAWINGS

FIGS. 1A-1D show that bacterial lactonase degrades patulin in vitro, inhibits apples' colonization, and inhibits gene expression of P. expansum patulin biosynthetic cluster in colonized apples. (1A) Michaelis-Menten kinetics analysis tested with 0.3 μM of bacterial lactonase (PPH-G55V) and patulin at pH 7.5, 25° C. (1B) The addition of 2 μM PPH-G55V to P. expansum spores resulted in reduced colonized area (upper panel, non-infected apples; mid panel, apples infected with P. expansum cultures; and lower panel, apples infected with P. expansum after cultures incubation with 2 μM PPH-G55V). Pictures were taken three days post-infection. (1C) Lesion size in cm² of treated apples after 3 days inoculation with P. expansum. Mean values are presented (*** p<0.0005, ** p<0.0047 according to one-way ANOVA followed by Tukey-Kramer). (1D) The relative expression levels of patulin biosynthesis pathway genes in infected apples following PPH-G55V enzymatic treatment were normalized to the housekeeping gene 28 s at the leading edge of the decay observed after 6 days of inoculation. Data points represent the means of three biological replicates ±standard error. Statistical analysis according to one-way ANOVA (p<0.05 *, <0.003 **). Fungi treated with the enzyme activity buffer was used as control in gray. ns—not significant.

FIGS. 2A-2D show that purified stabilized bacterial lactonase (PPH-G55V) reduced mycelium production and modulated its morphology in PDB medium. (2A) The addition of 2 μM PPH-G55V bacterial lactonase to a PDB medium containing ˜2500 spores of P. expansum, reduced mycelium production after three days (right tube), compared with untreated culture (left tube). (2B) Microscopic picture (×10), presenting the differences in fungal mycelia development between untreated mycelia (left) and PPH-G55V-treated mycelia (right). (2C) Fungal mycelium fresh weight was significantly lower in the presence of the lactonase than untreated fungi. (** p=0.0090, t test) (2D) Expression levels of genes (Gel and Bgt) normalized to housekeeping gene 28 s. Data points represent the means of three biological replicates±SE. Statistical significance according to one-way ANOVA comparison (p=0.0267 * left; p=0.0435 * right). Fungi treated with enzyme activity buffer used as control.

FIG. 3 shows the identification of putative lactonases in various fungal species based on sequence homology and structural modeling of P. expansum homolog. Multiple-sequence alignment of newly identified putative fungal lactonases. The color intensity correlates with the percentage identity. The HxHxDH˜H˜D˜H motif is common to all AHL lactonases in the metallo-β-lactamase (MBL) superfamily. The first sequence is of the homolog from P. expansum. The residues that coordinate the two catalytic metals are marked. The structural homology model of the putative lactonase from P. expansum, PELa indicated structural similarity to an AHL lactonase from Alicyclobacillus acidoterrestris (pdb 36cgy).

FIGS. 4A-4C show biochemical characterization of P. expansum newly identified enzyme (PELa). The activity of recombinantly expressed and purified PELa from P. expansum was tested at different pH levels (4A) and different temperatures (4B). Error bars indicate standard deviation of three replicates for each treatment. (4C) Michaelis-Menten kinetics analysis with 0.6 μM of fungal lactonase in activity buffer, 100 mM Tris-HCl pH 7.5, 100 mM M NaCl, and 100 μM ZnCl₂, and 0-0.4 mM patulin in activity buffer pH 7.5, at 25° C.

FIG. 5 shows lactone-based patulin and AHLs in fungal and bacterial species. The ability of lactonase to degrade these lactones and affect gene expression and virulence in both bacteria and fungi; suggest patulin degradation by lactonases might have an ecological role in both fungal and bacterial species.

FIGS. 6A-6B shows the enzymatic activity of purified E. amylovora lactonase (EaAiiA) in the presence of patulin in vitro and its contribution to the inhibition of P. expansum colonization in apple fruit. (6A) Michaelis-Menten kinetics analysis with 0.05 μM purified bacterial lactonase (EaAiiA), in activity buffer containing 100 mM Tris pH 7.5, 100 mM NaCl, 100 μM ZnCl₂, at Error bars indicate standard deviation of three replicates for each treatment. (6B) Colonization of P. expansum by mix-inoculation of active or inactive 2 μM EaAiiA. The lesion size of the treated apples was measured 3 days after inoculation with P. expansum. Mean values of 18 replicates are presented (**** p<0.0001, according to one-way ANOVA followed by Tukey-Kramer).

### DETAILED DESCRIPTION OF PREFERRED EMBODIMENTS

WO 2020/255131 of the same applicant discloses M. tuberculosis phosphotriesterase-like lactonase mutants having an improved stability, and the use of those mutants as well as the wild-type phosphotriesterase-like lactonase in treating or preventing infection of plants by bacterium secreting a lactone selected from N-(3-hydroxybutanoyl)-L-homoserine lactone (C4-HSL), N-(3-oxo-hexanoyl)-homoserine lactone (C6-oxo-HSL), N-[3S)-tetrahydro-2-oxo-3-furanyl]octanamide (C8-oxo-HSL), and N-[(3S)-tetrahydro-furanyl]decanamide (C10-HSL). The efficiency of such biocontrol agents has not been tested on fungal pathogens.

It has now been found, in accordance with the present invention, that purified bacterial phosphotriesterase-like lactonase effectively inhibits P. expansum infection in apples. As further found, the phosphotriesterase-like lactonase is capable of hydrolyzing patulin (4-hydroxy-4H-furo[3,2-c]pyran-2(6H)-one), a mycotoxin secreted by P. expansum.

As shown herein, the enzyme presented an inhibitory effect on P. expansum cultures when applied before apple infection, including downregulation of genes expression. To maintain enzymes' stability upon its addition to fungal cultures and during infection, a stabilized mutant of parathion protein hydrolase (PPH) (Zhang et al., 2019) from Mycobacterium tuberculosis was used. PPH-G55V presented improved residual activity at high temperatures (Gurevich et al., 2021), and it is therefore more suitable for biotechnological applications, testing lactonases activity, and their effects in cultures. The experimental section herein further shows the identification and characterization of a new lactonase from P. expansum, which is active with patulin. The data presented indicate a possible role for patulin and its degradation by lactonases in inter-kingdom communication between fungi and bacteria, and further suggest quorum-quenching lactonases as potential antifungal post-harvest treatment as a strategy to lower fungal mycotoxins food contamination.

A further study conducted has shown that similar effects were achieved using the AHL lactonase encoded by Erwinia amylovora (EaAiiA), previously identified as a homolog to AHL lactonase possessing the metallo-β-lactamase fold (Ya'ar Bar et al., 2021).

In one aspect, the present invention thus provides a method (also referred to herein “Method A”) for treating or preventing infection of a fungus in a plant or a part, organ or a propagation material thereof, or in a product made from said plant, part, organ or propagation material, said fungus secreting patulin, and said method comprising applying a lactonase or a functional fragment thereof on said plant, part, organ or propagation material; or to said product.

In another aspect, the present invention provides a method (also referred to herein “Method B”) for reducing the concentration of patulin in a plant or a part, organ or a propagation material thereof; in a product made from said plant, part, organ or propagation material; or in a non-plant food product, said method comprising applying a lactonase or a functional fragment thereof on said plant, part, organ or propagation material; or to said product.

In certain embodiments, the lactonase used according to any one of the methods disclosed herein is the wild-type putative parathion hydrolase from M. tuberclorosis (PPH; SEQ ID NO: 1).

In certain embodiments, the lactonase used according to any one of the methods disclosed herein is a phosphotriesterase (PTE)-like lactonase having at least 30% identity to wild-type putative parathion hydrolase from M. tuberclorosis (PPH; SEQ ID NO: 1, Table 1), a TIM-barrel fold substantially identical to that of the wild-type PPH, and preserved catalytic residues in its active site.

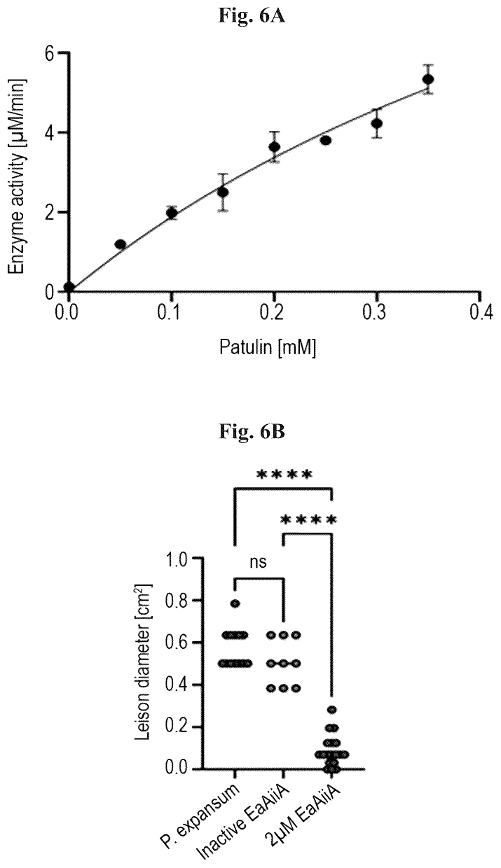

Phosphotriesterase-like lactonase from M. tuberculosis (PPH) is a quorum quenching enzyme, which belongs to the phosphotriesterase-like lactonases (Afriat et al., 2006) possessing a TIM barrel fold and preserved catalytic site as defined below. The location of a certain amino acid residue in the proteins or fragments thereof disclosed herein is according to the numbering of the wild type M. tuberculosis phosphotriesterase-like lactonase as depicted in SEQ ID NO: 1 and is designated by referring to the one-letter code of the amino acid residue and its position in the wild type M. tuberculosis phosphotriesterase-like lactonase. Thus, for example, the glycine at the position corresponding to position 59 of the wild type M. tuberculosis phosphotriesterase-like lactonase, also referred to herein as G59, would be referred to as G59 also in a phosphotriesterase-like lactonase fragment or in a homologous phosphotriesterase-like lactonase of a different size according to alignment algorithms well known in the art of protein chemistry, such as Multiple Sequence Comparison by Log-Expectation (MUSCLE) or Multiple Alignment using Fast Fourier Transform (MAFFT) (see, e.g., FIG. 6 in WO 2020/255131).

For clarity, the position of the amino acid residues in the sequences of the fusion-proteins used in the Examples section below, G55, corresponds to G59 in the isolated wild-type full length protein. Similarly, the sequence of the functionally active deletion mutant used to solve the three-dimensional structure of the phosphotriesterase-like lactonase from M. tuberculosis lacks the four first N-terminal amino acid residues (Zhang et al., 2019). Consequently, glycine at position 55 in the enzyme characterized in this specification corresponds to G59 according to the system used to identify amino acid residue positions in the enzymes of the present invention.

A substitution of an amino acid residue at a certain position with another amino acid residue is designated by referring to the one-letter code of the original amino acid residue, its position as defined above and the one-letter code of the amino acid residue replacing the original amino acid residue. Thus, e.g., a substitution of G59 with valine would be designated G59V.

The proteins encoded by the nucleic acid molecules of the invention are not limited to those defined herein by specific amino acid sequences but may also be variants of these proteins or have amino acid sequences that are substantially identical to those disclosed herein. A “substantially identical” amino acid sequence as used herein refers to a sequence that differs from a reference sequence by one or more conservative or non-conservative amino acid substitutions, deletions, or insertions, particularly when such a substitution occurs at a site that is not the active site of the nucleic acid molecule, and provided that the polypeptide encoded by said sequence essentially retains the functional properties of the polypeptide encoded by said reference sequence.

A conservative amino acid substitution, for example, substitutes one amino acid with another of the same class, e.g., substitution of one hydrophobic amino acid with another hydrophobic amino acid, a polar amino acid with another polar amino acid, a basic amino acid with another basic amino acid, and an acidic amino acid with another acidic amino acid. One or more consecutive amino acids can be deleted from either or both the N- and C-terminus of the peptide, thus obtaining a fragment of said peptide having a biological activity substantially identical to that of the peptide, referred to herein as a “functional fragment”. In contrast, deletion of one or more non-consecutive amino acids from the nucleic acid molecule may result in a “variant” of said peptide. The term “variant” as used herein refers to polynucleotides or polypeptides modified at one or more base pairs, codons, or amino acid residues, respectively, yet retaining the biological and enzymatic activity of a polypeptide of the naturally occurring sequence.

In certain embodiments, the biological activity or enzymatic function of all mutated phosphotriesterase-like lactonases including all variants and homologs are defined by substrate specificity and kinetic parameters, such as k_{cat}, K_{M} and k_{cat}/K_{M}. Methods for measuring lactonase activity are well known in the art; for example, the hydrolysis of a lactone such as C6-oxo-homoserine lactone can be monitored by following the appearance of the carboxylic acid products using a pH indicator as described in Afriat et al., 2006.

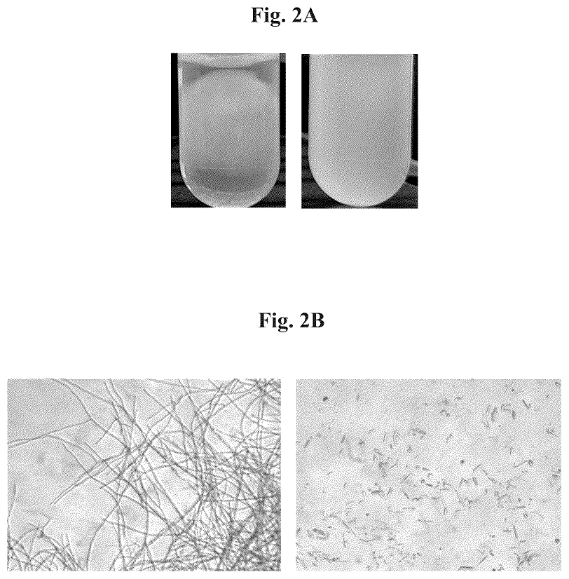

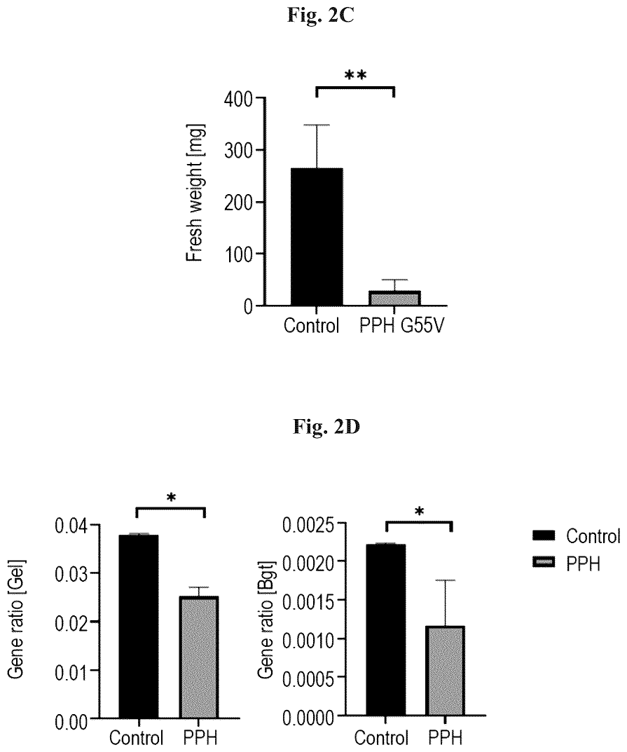

The catalytic residues are conserved throughout the PTE like lactonases: His26, His28, His182 and His211, and Asp270. The sixth ligating residue is a carbamylated Lys149, (numbering are for PPH) (see FIG. 2D and FIG. 6 in WO 2020/255131). A mutation in any one of these amino acid residues leads to loss of function. Consequently, as defined above, any one of the mutated phosphotriesterase-like lactonases used in the present invention has an intact active site, i.e., each one of the amino acid residues of these mutated phosphotriesterase-like lactonases corresponding to His26, His28, Lys149, His182, His211 and Asp270 in the wild-type full length PPH of SEQ ID NO: 1 is conserved.

In certain embodiments, the lactonases used according to any one of the methods disclosed herein are thus phosphotriesterase-like lactonases, including the wild-type putative parathion hydrolase from M tuberclorosis (PPH; SEQ ID NO: 1) as well as homologues, variants and mutants of said PPH, having at least 30% identity with SEQ ID NO: 1 and a TIM-barrel fold that is substantially identical to that of the wild-type enzyme, capable of hydrolyzing lactones such as C4-HSL (PubChem CID: 10330086 aka 3-hydroxy-C4-HSL, N-(3-hydroxybutanoyl)-L-homoserine lactone), C6-oxo-HSL (PubChem CID, 688505, aka N-(3-oxo-hexanoyl)-homoserine, N-caproyl-L-homoserine lactone, N-[(3S)-tetrahydro-2-oxo-3-furanyl]hexanamide, HHL), C8-oxo-HSL (PubChem CID: 6914579 aka N-[(3S)-tetrahydro-2-oxo-3-furanyl]octanamide) and C10-HSL (PubChem CID: 10131281 aka N-[3S)-tetrahydro-2-oxo-3-furanyl]decanamide), and C6-oxo-HSL.

The term “TIM-barrel fold” is used herein in its conventional meaning and refers to a conserved protein fold consisting of eight α-helices and eight parallel (3-strands that alternate along the peptide backbone (Wierenga RK., 2001). Methods for determining the tertiary structure of a protein or generating a model thereof are well-known in the art and can easily be done for a large number of proteins. For example, a model for determining the TIM-barrel fold may be generated using ModPipe, an automated software, pipeline, that calculates models on the basis of known structural templates and sequence-structure alignments (Pieper et al., 2011).

The variants and homologs of the mutated wild-type phosphotriesterase-like lactonase used according to any one of the methods disclosed herein are defined by their sequence identity with the wild-type phosphotriesterase-like lactonase of SEQ ID NO: 1, not including the mutation characterizing the mutant protein. Thus, for example, a homolog having 90% identity with the mutant G59V has 90% identity with the sequence including amino acid residues 1-58 and 60-330 (or with the sequence including amino acid residues 1-330 and relating to position 59 as identical to wild-type G59).

In certain embodiments, the amino acid sequence having at least 30% identity with SEQ ID NO: 1 has 30%-99%, 30%-98%, 30%-97%, 30%-96%, 30%-95%, 30%-90%, 30%-85%, 30%-80%, 30%-75%, 30%-70%, 30%-65%, 30%-60%, 30%-55%, 30%-50%, 30%-45%, 30%-40%, 40%-99%, 40%-98%, 40%-97%, 40%-96%, 40%-95%, 40%-90%, 40%-85%, 40%-80%, 40%-75%, 40%-70%, 40%-65%, 40%-60%, 40%-55%, 40%-50%, 40%-45%, 50%-99%, 50%-98%, 50%-97%, 50%-96%, 50%-95%, 50%-90%, 50%-85%, 50%-80%, 50%-75%, 50%-70%, 50%-65%, 50%-60%, 50%-55%, 60%-99%, 60%-98%, 60%-97%, 60%-96%, 60%-95%, 60%-90%, 60%-85%, 60%-80%, 60%-75%, 60%-70%, 60%-65%, 70%-99%, 70%-98%, 70%-97%, 70%-96%, 70%-95%, 70%-90%, 70%-85%, 70%-80%, 70%-75%, 80%-99%, 80%-98%, 80%-97%, 80%-96%, 80%-95%, 80%-90%, 80%-85%, 90%-99%, 90%-98%, 90%-97%, 90%-96%, or 90%-95% identity with SEQ ID NO: 1.

In certain embodiments, the amino acid sequence having at least 30% identity with SEQ ID NO: 1 has at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least at least 81, at least 82, at least 83, at least 84, at least 85, at least 86, at least 87, at least 88, at least 89, at least 90, at least 91, at least 92, at least 93, at least 94, at least 95, at least 96, at least 97, at least 98%, or at least 99% identity with SEQ ID NO: 1.

In certain embodiments, the amino acid sequence having at least 30% identity with SEQ ID NO: 1 has 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity with SEQ ID NO: 1. In certain embodiments, the amino acid sequence has at least 79% identity with SEQ ID NO: 1 and the protein encoded by said sequence is selected from the group of proteins herein identified Proteins 1-92 in Table 2.

In certain embodiments, a glycine residue corresponding to G59 of SEQ ID NO: 1 is substituted by valine, alanine, leucine, or isoleucine; or a histidine residue corresponding to H172 of SEQ ID NO: 1 is substituted by tyrosine phenylalanine or tryptophan. In certain particular such embodiments, any one of these substitutions is the sole substitution in the sequence of the mutated phosphotriesterase-like lactonase as compared with the sequence of the corresponding wild-type protein, e.g., a protein selected from Proteins 1-92 in Table 2, except for optional conservative substitutions of other amino acid residues or optional deletion of one or more amino acid residues at the N- or C-terminus. In other particular such embodiments, any one of these substitutions is the sole substitution in the sequence of the mutated phosphotriesterase-like lactonase as compared with SEQ ID NO: 1, i.e., no other modifications are made to the amino acid sequence, except for optional deletions of amino acid residues, e.g., at the N- or C-terminus that do not affect enzymatic function.

In certain embodiments, a glycine residue corresponding to G59 of SEQ ID NO: 1 is substituted by valine. In certain particular such embodiments, any one of these substitutions is the sole substitution in the sequence of the mutated phosphotriesterase-like lactonase as compared with the sequence of the corresponding wild-type protein, e.g., a protein selected from Proteins 1-92 in Table 2, except for optional conservative substitutions of other amino acid residues or optional deletion of one or more amino acid residues at the N- or C-terminus. In other particular such embodiments, this is the sole substitution in the sequence of the mutated phosphotriesterase-like lactonase as compared with SEQ ID NO: 1, except for conservative substitutions of other amino acid residues. In further particular such embodiments, this is the sole substitution in the sequence of the mutated phosphotriesterase-like lactonase as compared with SEQ ID NO: 1, i.e., no other modifications are made to the amino acid sequence, except for optional deletion of one or more amino acid residues at the N- or C-terminus. In yet other particular such embodiments, the mutated phosphotriesterase-like lactonase comprises or essentially consists of the amino acid sequence as set forth in SEQ ID NO: 2 (Table 1).

In certain embodiments, a histidine residue corresponding to H172 of SEQ ID NO: 1 is substituted by tyrosine. In certain particular such embodiments, any one of these substitutions is the sole substitution in the sequence of the mutated phosphotriesterase-like lactonase as compared with the sequence of the corresponding wild-type protein, e.g., a protein selected from Proteins 1-92 in Table 2, except for optional conservative substitutions of other amino acid residues or optional deletion of one or more amino acid residues at the N- or C-terminus. In other particular such embodiments, this is the sole substitution in the sequence of the mutated phosphotriesterase-like lactonase as compared with SEQ ID NO: 1, except for conservative substitutions of other amino acid residues. In further particular such embodiments, this is the sole substitution in the sequence of the mutated phosphotriesterase-like lactonase as compared with SEQ ID NO: 1, i.e., no other modifications are made to the amino acid sequence, except for optional deletion of one or more amino acid residues at the N- or C-terminus. In yet other particular such embodiments, the mutated phosphotriesterase-like lactonase comprises or essentially consists of the amino acid sequence as set forth in SEQ ID NO: 3 (Table 1).

In other embodiments, the lactonase used according to any one of the methods disclosed herein is an acyl-homoserine (AHL) lactonase such as 1,4-lactonase (EC 3.1.1.25), 2-pyrone-4,6-dicarboxylate lactonase, 3-oxoadipate enol-lactonase, actinomycin lactonase, deoxylimonate A- ring-lactonase, gluconolactonase, L-rhamnono-1,4-lactonase, limonin-D-ring-lactonase, steroid-lactonase, triacetate-lactonase, and xylono-1,4-lactonase. Particular such lactonases may possess different folds, e.g., a triose-phosphate isomerase (TIM)-barrel fold substantially identical to that of the wild-type PPH, a metallo-β-lactamase (MBL) fold such as in the case of EaAiiA, or six-bladed β-propeller such as in the case of the paraoxonase (PON) family

The term “MBL fold” is used herein in its conventional meaning and refers to the fold of AHL lactonases from the AiiA-type metallohydrolases of the β-lactamase family (MBLs), which possesses an α/β hydrolase fold and requires two Zn²⁺ ions in the active site for full functionality.

In certain embodiments, the lactonase used according to any one of the methods disclosed herein is the wild-type putative AHL lactonase from E. amylovora (EaAiiA; SEQ ID NO: 7, Table 1), which has the MBL fold.

In certain embodiments, the lactonase used according to any one of the methods disclosed herein is a lactonase having at least 30% identity to wild-type putative AHL lactonase from E. amylovora (SEQ ID NO: 7), a MBL fold substantially identical to that of the wild-type putative AHL lactonase from E. amylovora, and preserved catalytic residues in its active site.

In certain embodiments, the amino acid sequence having at least 30% identity with SEQ ID NO: 7 has 30%-99%, 30%-98%, 30%-97%, 30%-96%, 30%-95%, 30%-90%, 30%-85%, 30%-80%, 30%-75%, 30%-70%, 30%-65%, 30%-60%, 30%-55%, 30%-50%, 30%-45%, 30%-40%, 40%-99%, 40%-98%, 40%-97%, 40%-96%, 40%-95%, 40%-90%, 40%-85%, 40%-80%, 40%-75%, 40%-70%, 40%-65%, 40%-60%, 40%-55%, 40%-50%, 40%-45%, 50%-99%, 50%-98%, 50%-97%, 50%-96%, 50%-95%, 50%-90%, 50%-85%, 50%-80%, 50%-75%, 50%-70%, 50%-65%, 50%-60%, 50%-55%, 60%-99%, 60%-98%, 60%-97%, 60%-96%, 60%-95%, 60%-90%, 60%-85%, 60%-80%, 60%-75%, 60%-70%, 60%-65%, 70%-99%, 70%-98%, 70%-97%, 70%-96%, 70%-95%, 70%-90%, 70%-85%, 70%-80%, 70%-75%, 80%-99%, 80%-98%, 80%-97%, 80%-96%, 80%-95%, 80%-90%, 80%-85%, 90%-99%, 90%-98%, 90%-97%, 90%-96%, or 90%-95% identity with SEQ ID NO: 7.

In certain embodiments, the amino acid sequence having at least 30% identity with SEQ ID NO: 7 has at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least at least 81, at least 82, at least 83, at least 84, at least 85, at least 86, at least 87, at least 88, at least 89, at least 90, at least 91, at least 92, at least 93, at least 94, at least 95, at least 96, at least 97, at least 98%, or at least 99% identity with SEQ ID NO: 7.

In certain embodiments, the amino acid sequence having at least 30% identity with SEQ ID NO: 7 has 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity with SEQ ID NO: 7.

For practical purposes, any one of the wild-type or mutated lactonases used according to the methods of the present invention may be provided as a fusion protein containing a tag useful for separating it from the cell extract by specific binding to a ligand-containing substrate or for improving solubility. For example, any one of the mutated lactonases used may be provided as a fusion protein with a maltose binding protein at the amino terminus. Other examples of tags include chitin binding protein (CBP), strep-tag (e.g., a selected nine-amino acid peptide (AWRHPQFGG) that displays intrinsic binding affinity towards streptavidin), glutathione-S-transferase (GST), and poly(His) tag. Tags including thioredoxin (TRX) and poly(NANP), used to improve solubility of the mutated phosphotriesterase-like lactonase, may also be used. The tag is optionally removable by chemical agents or by enzymatic means, such as proteolysis or intein splicing.

Alternatively, the lactonase may be provided or encoded as a fusion protein containing a signal sequence facilitating its secretion into the growth medium. This is useful because it eliminates the need for disrupting the cells and provides for harvesting the protein of the invention simply by collecting the growth medium. The signal sequence is tailored for the host cell type used to express the protein. Freudl (2018) teaches that, in bacteria, two major export pathways, the general secretion or Sec pathway and the twin-arginine translocation or Tat pathway, exist for the transport of proteins across the plasma membrane. The routing into one of these alternative protein export systems requires the fusion of a Sec- or Tat-specific signal peptide to the amino-terminal end of the desired target protein.

In short, the lactonase used according to any one of the methods disclosed herein may be provided as a fusion protein containing a Sec or Tat signal peptide. These peptides possess a similar tripartite overall structure consisting of a positively charged n-region, a central hydrophobic h-region, and a polar c-region that contains the recognition site (consensus: A-X-A) for signal peptidase. In Tat signal peptides, a characteristic amino acid consensus motif including two highly conserved arginine residues is present at the boundary between the often significantly longer n-region and the h-region. Furthermore, the h-region of Tat signal peptides is mostly less hydrophobic than those found in Sec signal peptides and in the c-region of Tat signal peptides, frequently positively charged amino acids (the so-called Sec-avoidance motif) are present that prevent a mistargeting of Tat substrates into the Sec pathway.

Since signal peptides, besides being required for the targeting to and membrane translocation by the respective protein translocases, also have additional influences on the biosynthesis, the folding kinetics, and the stability of the respective target proteins, so far it is not possible to predict in advance which signal peptide will perform best in the context of a given target protein and a given bacterial expression host. However, methods for finding an optimal signal peptide for a desired protein are well known and are described, e.g., in Freudl (2018). The signal sequence may be removed during the process of secretion, or it is optionally removable by chemical agents or by enzymatic means, such as proteolysis or intein splicing.

In certain embodiments, any one of the mutated lactonases used according to the methods of the present invention, when fused to a tag, may lack 1 to 10 amino acid residues at its N- or C-terminus (as compared with the corresponding wild-type), such as 1-4 amino acid residues at the N-terminus and said tag is fused to the N-terminus. Furthermore, a linker may be inserted between the sequence of the tag and the mutated lactonases, such as a poly-asparagine of, e.g., about 10 residues.

In certain embodiments, the mutated phosphotriesterase-like lactonases fusion protein is of SEQ ID NO: 4, 5 or 6 (Table 1).

In certain embodiments, the mutated phosphotriesterase-like lactonase used according to the methods of the present invention has an increased thermostability in comparison with the thermostability of a non-mutated wild-type phosphotriesterase-like lactonase and/or substantially similar or higher lactonase catalytic activity provided with N-(3-oxo-hexanoyl)-homoserine lactone (C6-oxo-HSL) as a substrate in comparison with said non-mutated wild-type phosphotriesterase-like lactonase.

The term “thermostability” as used herein refers to the inherent property of a protein of maintaining its activities at or after being exposed to high temperatures, i.e., temperatures that causes partial or total denaturation and loss of activity in most related proteins. The thermostability is often measured in a relative term, T₅₀, representing the temperature at which 50% of the enzyme's maximal activity (at optimal conditions) is obtained after incubating the enzyme in a range of temperatures and then measuring catalytic activity at optimal temperature, referred to herein as “50% residual activity”.

In certain embodiments, the increased thermostability is characterized by 50% residual activity (following incubation at a certain temperature) that is substantially or significantly higher than that of the wild type phosphotriesterase-like lactonase, i.e., at a temperature substantially or significantly higher than about 40° C.

In certain embodiments, the increased thermostability expressed as 50% residual activity (T₅₀) is at about 50° C.-80° C., 50° C.-75° C., 50° C.-70° C., 50° C.-65° C., 60° C.-80° C., 60° C.- 75° C., 60° C.-60° C.-65° C., 70° C.-80° C., 70° C.-75° C., or 75° C.-80° C.; or at 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.

In certain embodiments, the increased thermostability comprises 50% residual activity at about 65° C. As shown in WO 2020/255131, a substitution of G59 to valine results in an enzyme with 50% residual activity at about 62° C., and a substitution of H172 to tyrosine results in an enzyme with 50% residual activity at about 65° C.

In certain embodiments, the mutated phosphotriesterase-like lactonase G59V results in an enzyme with a k_{cat}/K_{M} that is twofold higher than that of the wild-type enzyme.

The term “substantially similar lactonase catalytic activity” as used herein refers to a lactonase activity that is in the same order of magnitude as that of the reference, e.g., in the same order of magnitude as the lactonase activity of the wild-type enzyme.

In certain embodiments, the mutated phosphotriesterase-like lactonase used according to the methods disclosed herein comprises or essentially consists of the amino acid sequence as set forth in SEQ ID NO: 2 or SEQ ID NO: 3; said increased thermostability expressed as T₅₀ is about to about 80° C., e.g., about 65° C.; and/or said mutated phosphotriesterase-like lactonase has an extended shelf-life as compared with said non-mutated phosphotriesterase-like lactonase.

Method A disclosed herein is aimed at treating or preventing infection of a fungus secreting patulin in a plant or a part, organ or a propagation material thereof, or in a product made from said plant, part, organ or propagation material.

In certain embodiments, the fungus treated with Method A is of a genus selected from Penicillium, e.g., Penicillium expansum, Aspergillus and Byssochlamys.

In other embodiments, the plant treated with Method A is selected from apple tree, cherry tree, blueberry shrub, plum tree, banana tree, strawberry bush, grape vine, barley grain, wheat grain, and corn grain; and said plant organ is a fruit of said plant. In particular embodiments, said plant is apple tree, and said fruit is apple.

In further embodiments, the product treated with Method A is selected from sauce, juice, jam, or an alcoholic beverage, made from said fruit; and barley, wheat or corn flour.

Method B disclosed herein is aimed at reducing the concentration of patulin in a plant or a part, organ or a propagation material thereof; in a product made from said plant, part, organ or propagation material; or in a non-plant food product.

In certain embodiments, the plant treated with Method B is selected from apple tree, cherry tree, blueberry shrub, plum tree, banana tree, strawberry bush, grape vine, barley grain, wheat grain, and corn grain; and said plant organ is a fruit of said plant. Particular embodiments are those wherein said plant is apple tree, and said fruit is apple.

In other embodiments, the product treated with Method B is selected from sauce, juice, jam, or an alcoholic beverage, made from said fruit.

In further embodiments, the non-plant food product treated with Method B is shellfish.

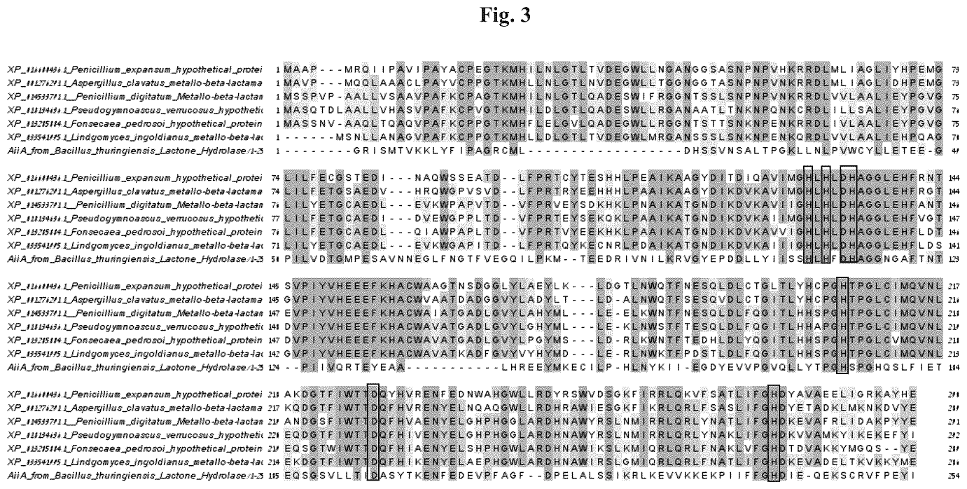

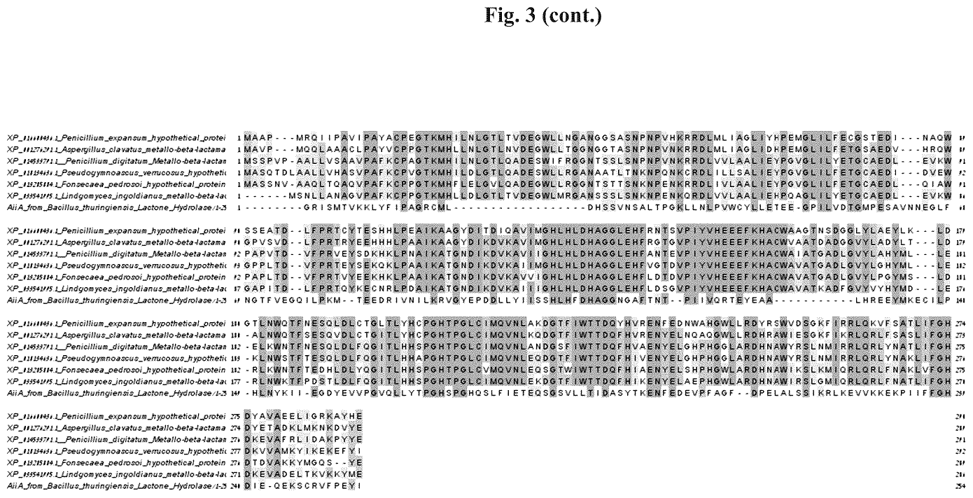

The term “treating” as used herein refers to means of obtaining a desired physiological effect. The effect may be therapeutic in terms of partially or completely curing a disease and/or symptoms attributed to the disease. The term refers to inhibiting the disease, i.e., arresting its development; ameliorating the disease, i.e., causing regression of the disease; or protecting a plant or a part, organ or a plant propagation material thereof from the disease by preventing or limiting infection. The term “treating” as used herein further refers to reduction of bacterial virulence as exhibited, e.g., in reduced extracellular polysaccharide (EPS) matrix or levan that contribute to the formation of the EPS (see FIG. 3 in WO 2020/255131).

The term “preventing” may be used herein interchangeably with the term “protecting” or “prophylactic treatment” and refers to application of a lactonase as defined in any one of the embodiments above, a functional fragment thereof, or a composition comprising said lactonase or said fragment, to a susceptible plant or a part, organ or a plant propagation material thereof, prior to discernible microbial infection.

A method of preventing infection of a fungus secreting patulin on, e.g., a seed, fruit, blossom, or flower, by applying a lactonase as defined in any one of the embodiments above, a functional fragment thereof, or a composition comprising said lactonase or said fragment, may result in subsequent reduced infection as compared with a seed, fruit, blossom, or flower that was not subject to this method of prevention, and the term “preventing” should thus not be understood as necessarily resulting in the total absence of microbial infection or microbial presence, since the treatment neither kills the bacteria nor inhibits cell growth. The effect of Method A may be observed, e.g., in the case of seeds that have been subject to the method of preventing microbial infection prior to discernible infection, which subsequent to planting yield plants having higher stem length and foliage mass as compared to plants derived from seeds that have not been subject to this method. The difference in plant biomass yield is a result of the absence of infection, or reduced level of infection in the pretreated seeds that developed subsequent and in spite of the prophylactic treatment, as compared with the non-treated seeds. Flowers, whole blossoms and fruit may similarly be pretreated by application of said lactonase, functional fragment thereof, or composition, which results in preservation of flower, blossom and fruit integrity and thus increased yield. Another example would be using the method of the present invention for preventing infection of a microorganism in a plant or seedling growing in the vicinity of infected plants (from the same field or from other fields). In case the infective agent spreads from the infected plants or field to the initially non-infected plants or field, prophylactic treatment will protect the plants and thus result in higher yield as compared with plants or seedlings that have not been subject to this method.

The methods of the present invention may comprise direct application of a lactonase as defined in any one of the embodiments above, a functional fragment thereof, or a composition comprising said lactonase or said fragment, to the plant or part, organ or plant propagation material thereof, or said lactonase, functional fragment thereof, or composition may be applied thereto in a formulation such as granules, dusts, emulsifiable concentrates, wettable powders, pastes, water-based flowables, dry flowables, oil agents, aerosols, fogs, or fumigants, with suitable solid carriers, liquid carriers, emulsifying and dispersing agents, etc.

In certain embodiments, any one of the compositions or formulations described above is applied to the plant or a part, organ or a plant propagation material thereof by spraying, immersing, dressing, coating, pelleting, or soaking.

In certain embodiments, Method A is for treating or preventing infection of a fungus secreting patulin on a propagation material such as a seed, root, fruit, tuber, bulb, rhizome, or part of a plant, wherein the lactonase, functional fragment thereof, or composition comprising it, is applied to the propagation material by spraying, immersing, dressing, coating, pelleting, or soaking prior to or after detection of the infection.

In certain embodiments, the part of a plant is a leaf, branch, flower, blossom, inflorescence, or a stem. In other embodiments, the plant organ is a fruit. In further embodiments, the plant propagation material is a seed or a fruit.

The term “phosphotriesterase-like lactonase from M. tuberculosis” is used interchangeably herein with the term “putative parathion hydrolase (PPH) from M. tuberculosis” and quorum quenching PPH.

The transition phrase “consisting essentially of” or “essentially consisting of”, when referring to an amino acid or nucleic acid sequence, refers to a sequence that includes the listed sequence and is open to present or absent unlisted sequences that do not materially affect the basic and novel properties of the protein itself, or the protein encoded by the nucleic acid sequence.

The term “substantially higher than” when referring to a temperature at which 50% residual activity is measured, refers to a difference of at least 5° C. higher than the reference.

The term “significantly higher than” refers to a statistically significant difference as tested with, e.g., Student's t-test with α=0.05.

TABLE 1
Protein sequences of wild-type and mutant PPH

| Sequence ID number | Sequence type | Comment |
|---|---|---|
| SEQ ID NO: 1 | Protein | wild type PPH (CKQ82621.1) |
| SEQ ID NO: 2 | Protein | G59V PPH |
| SEQ ID NO: 3 | Protein | H172Y PPH |
| SEQ ID NO: 4 | Protein | wild type PPH-MBP fusion* |
| SEQ ID NO: 5 | Protein | G59V PPH-MBP fusion* |
| SEQ ID NO: 6 | Protein | H172Y PPH-MBP fusion* |
| SEQ ID NO: 7 | Protein | wild type EaAiiA |

*PPH is lacking the N-terminal methionine.

TABLE 2
Protein sequences of PPH homologs (PTE-like lactonases)

| Protein number | Accession number/Protein name | Protein source |
|---|---|---|
| 1 | CKS73406.1 parathion hydrolase | Mycobacterium tuberculosis |
| 2 | SGN98718.1 parathion hydrolase | Mycobacterium tuberculosis |
| 3 | AAK44461.1 parathion hydrolase | Mycobacterium tuberculosis CDC1551 |
| 4 | WP_003900835.1 PTE-related protein | Mycobacterium tuberculosis |
| 5 | WP_031702804.1 PTE-related protein | Mycobacterium tuberculosis |
| 6 | WP_070891680.1 PTE-related protein | Mycobacterium tuberculosis |
| 7 | WP_069334075.1 PTE | Mycobacterium tuberculosis |
| 8 | WP_003401263.1 Multispecies: PTE | Mycobacterium |
| 9 | WP_055366308.1 PTE | Mycobacterium tuberculosis |
| 10 | WP_057136094.1 PTE | Mycobacterium tuberculosis |
| 11 | WP_031672770.1 PTE family protein | Mycobacterium tuberculosis |
| 12 | WP_031726559.1 PTE-related protein | Mycobacterium tuberculosis |
| 13 | WP_031700829.1 PTE family protein | Mycobacterium tuberculosis |
| 14 | WP_031665946.1 PTE | Mycobacterium tuberculosis |
| 15 | WP_031687538.1 PTE family protein | Mycobacterium tuberculosis |
| 16 | WP_128884084.1 PTE-related protein | Mycobacterium tuberculosis |
| 17 | WP_057118862.1 PTE | Mycobacterium tuberculosis |
| 18 | WP_031751683.1 PTE family protein | Mycobacterium tuberculosis |
| 19 | WP_015629423.1 PTE-related protein | Mycobacterium tuberculosis |
| 20 | WP_015302462.1 PTE Php (parathion | Mycobacterium canettii |
| | hydrolase) (PTE) | |
| | (aryldialkylphosphatase) (paraoxonase) | |
| | (a-esterase) (aryltriphosphatase) | |
| | (paraoxon hydrolase) | |
| 21 | WP_070916822.1 PTE-related protein | Mycobacterium tuberculosis |
| 22 | WP_057370492.1 PTE | Mycobacterium tuberculosis |
| 23 | WP_041153720.1 PTE | Mycobacterium tuberculosis |
| 24 | WP_031751646.1 PTE | Mycobacterium tuberculosis |
| 25 | WP_031716625.1 PTE | Mycobacterium tuberculosis |
| 26 | WP_031707299.1 PTE | Mycobacterium tuberculosis |
| 27 | WP_052636504.1 PTE | Mycobacterium tuberculosis |
| 28 | WP_031711112.1 PTE | Mycobacterium tuberculosis |
| 29 | RYD10130.1 PTE | Mycobacterium tuberculosis |
| 30 | WP_017487637.1 PTE | Mycobacterium tuberculosis |
| 31 | WP_014585487.1 PTE | Mycobacterium tuberculosis |
| 32 | WP_102776491.1 PTE-related protein | Mycobacterium tuberculosis |
| 33 | WP_055384803.1 PTE | Mycobacterium tuberculosis |
| 34 | WP_057174556.1 PTE | Mycobacterium tuberculosis |
| 35 | 4IF2_A Chain A, structure of the PTE | |
| | from Mycobacterium tuberculosis | |
| 36 | WP_055374072.1 PTE | Mycobacterium tuberculosis |
| 37 | WP_031725478.1 PTE-related protein | Mycobacterium tuberculosis |
| 38 | WP_031738135.1 PTE | Mycobacterium tuberculosis |
| 39 | WP_014000125.1 PTE | Mycobacterium canettii |
| 40 | WP_052632536.1 PTE | Mycobacterium tuberculosis |
| 41 | WP_031752956.1 PTE | Mycobacterium tuberculosis |
| 41 | WP_015288873.1 PTE Php (parathion | Mycobacterium canettii |
| | hydrolase) (PTE) | |
| | (aryldialkylphosphatase) (paraoxonase) | |
| | (a-esterase) (aryltriphosphatase) | |
| | (paraoxon hydrolase) | |
| 42 | WP_050895789.1 PTE | Mycobacterium tuberculosis |
| 43 | WP_031652122.1 PTE | Mycobacterium tuberculosis |
| 44 | WP_052655401.1 PTE | Mycobacterium tuberculosis |
| 45 | WP_057136546.1 PTE | Mycobacterium tuberculosis |
| 45 | WP_013988719.1 PTE | Mycobacterium tuberculosis |
| 46 | WP_015291993.1 PTE Php (parathion | Mycobacterium canettii |
| | hydrolase) (PTE) | |
| | (aryldialkylphosphatase) (paraoxonase) | |
| | (a-esterase) (aryltriphosphatase) | |
| | (paraoxon hydrolase) | |
| 47 | AUS49258.1 PTE | Mycobacterium tuberculosis |
| 48 | SGD30548.1 parathion hydrolase | Mycobacterium tuberculosis |
| 49 | WP_049873613.1 PTE | Mycobacterium tuberculosis |
| 50 | WP_085159921.1 PTE-related protein | Mycobacterium lacus |
| 51 | WP_009979649.1 Multispecies: PTE | Mycobacterium avium complex |
| 52 | WP_016810152.1 PTE | Mycobacterium tuberculosis |
| 53 | WP_054878907.1 PTE | Mycobacterium haemophilus |
| 54 | WP_063470385.1 Multispecies: PTE | Mycobacterium |
| 55 | WP_069397147.1 PTE | Mycobacterium shimoidei |
| 55 | WP_113963099.1 PTE-related protein | Mycobacterium shimoidei |
| 56 | WP_085182214.1 PTE -related protein | Mycobacterium bohemicum |
| 57 | WP_075542160.1 PTE | Mycobacterium kansasii |
| 58 | WP_003874067.1 PTE | Mycobacterium avium |
| 59 | WP_082966984.1 PTE-related protein | Mycobacterium sp. 852002- |
| | | 51163_SCH5372311 |
| 60 | VDM86860.1 Parathion hydrolase | Mycobacterium sp. DSM 104308 |
| | precursor | |
| 61 | WP_047316850.1 PTE | Mycobacterium haemophilu |
| 62 | WP_075546659.1 PTE | Mycobacterium persicum |
| 63 | WP_122510178.1 PTE -related protein | Mycobacterium persicum |
| 64 | WP_023369760.1 Multispecies: PTE | Mycobacterium |
| 65 | WP_067372810.1 PTE | Mycobacterium sp. 1164966.3 |
| 66 | WP_094028596.1 PTE-related protein | Mycobacterium avium |
| 67 | WP_066917426.1 PTE | Mycobacterium interjectum |
| 68 | WP_122440715.1 Multispecies: | Mycobacterium |
| | PTE-related protein | |
| 69 | ORB95896.1 PTE-related protein | Mycobacterium persicum |
| 70 | WP_083124567.1 PTE-related protein | Mycobacterium kansasii |
| 71 | WP_085199107.1 PTE-related protein | Mycobacterium fragae |
| 72 | WP_068024441.1 PTE | Mycobacterium kubicae |
| 73 | WP_068157568.1 PTE | Mycobacterium kubicae |
| 74 | WP_068229952.1 PTE | Mycobacterium sp. E3198 |
| 75 | WP_085327573.1 PTE -related protein | Mycobacterium decipiens |
| 76 | WP_083116038.1 Multispecies: | Mycobacterium |
| | PTE-related protein | |
| 77 | WP_068061678.1 PTE | Mycobacterium sp. E342 |
| 78 | WP_067254020.1 PTE | Mycobacterium sp. 852002- |
| | | 10029_SCH5224772 |
| 79 | WP_036413589.1 PTE | Mycobacterium gastri |
| 80 | WP_085250078.1 PTE-related protein | Mycobacterium riyadhense |
| 81 | WP_046184118.1 PTE | Mycobacterium nebraskense |
| 82 | WP_103845650.1 PTE -related protein | Mycobacterium kansasii |
| 83 | WP_067099853.1 PTE | Mycobacterium sp. 852002- |
| | | 40037_SCH5390672 |
| 84 | WP_085072500.1 PTE-related protein | Mycobacterium kubicae |
| 85 | WP_117389070.1 PTE-related protein | Mycobacterium marinum |
| 86 | WP_065475716.1 PTE | Mycobacterium malmoense |
| 87 | WP_083178402.1 PTE-related protein | Mycobacterium scrofulaceum |
| 88 | WP_012392457.1 PTE | Mycobacterium marinum |
| 89 | WP_068094268.1 PTE | Mycobacterium sp. E2497 |
| 90 | WP_044509449.1 PTE | Mycobacterium simiae |
| 91 | WP_117431711.1 PTE-related protein | Mycobacterium marinum |
| 92 | WP_068140455.1 PTE | Mycobacterium sp. E796 |

The term “about” as used herein means that values which are up to 10% above or below the value provided are also included. Numbers that are not preceded by the term “about” are nevertheless to be understood as being modified in all instances by this term. Accordingly, unless indicated to the contrary, the numerical parameters set forth in this description and attached claims are approximations that may vary by up to plus or minus 10% depending upon the desired properties sought to be obtained by the present invention.

The invention will now be illustrated by the following non-limiting Examples.

### EXAMPLES

Study 1. Bacterial quorum-quenching lactonase hydrolyzes fungal mycotoxin and reduces pathogenicity of Penicillium expansum

### Materials and Methods

Fungal growth conditions. The plant pathogen P. expansum Pe-21 was isolated from decayed local cvs. Grand Alexander and Golden Delicious apples, provided by the Department of Postharvest Science of Fresh Produce, ARO, Volcani Center, Israel (Hadas et al., 2007). Isolate Pe-21 was used to study the activation of glucose oxidase and secretion of gluconic acid by P. expansum pathogenicity in apples (Hadas et al., 2007). Moreover, Pe-21 knockout established a connection between LaeA, a global regulator and the regulation of several secondary metabolite genes, including the patulin gene cluster (Kumar et al., 2017). Cultures were grown on potato dextrose agar (PDA) plates (Difco, Detroit, MI, USA) at room temperatures at the range of 22-24° C. in the dark. Mycelial growth and fruit inoculation were assays from one-week-old conidia, harvested from potato dextrose agar PDA plates. Conidia were harvested from PDA plates after adding 5 mL of sterile distilled water with 0.01% (v/v) Tween 20 (Sigma-Aldrich, Copenhagen, Denmark), gently rubbing the fungal spores, pulling the liquid together, and collected to 1.5 mL tubes. Potato dextrose broth (PDB) medium (Difco, Detroit, MI, USA) was used for growing liquid cultures. P. expansum spores are ellipsoidal, 3.0-3.5 μm long, and smooth-walled.

Recombinant expression and purification of lactonases. We used a variant of a highly efficient AHL lactonase, parathion protein hydrolase (PPH), from M. tuberculosis belonging to the phosphotriesterase (PTE)-like lactonase family (Zhang et al., 2019). We previously obtained PPH-G55V by using directed enzyme evolution (Gurevich et al., 2021). The variant harbors one point mutation (Gly to Val at position 55) exhibited increased thermal stability and shelf life (Gurevich et al., 2021), essential criteria for biotechnological applications. pMAL-c4X-PPH-G55V vector was used for lactonase expression as a fusion protein with maltose binding protein (MBP). Its recombinant expression and purification were performed as previously described (Gurevich et al., 2021). Briefly, freshly transformed E. coli-BL21 (DE3) cells with pMAL-c4xPPH-G55V, were inoculated in to 10 mL LB medium with 100 μg/mL ampicillin and 0.5 mM MnCl₂. Cultures were grown at 37° C., 170 rpm. Following overnight growth, cultures were added to 1 L LB medium for several hours at 30° C., 170 rpm. When the cultures OD₆₀ reached 0.6-0.8, expression was executed by the addition of 0.4 mM IPTG (isopropyl (3-d-1-thiogalactopyranoside) for overnight expression at 20° C. Cells were harvested by centrifugation, and then suspended in lysis buffer containing 100 mM Tris-HCl pH 8.0, 100 mM NaCl, 100 μM MnCl₂ and protease inhibitor cocktail (Sigma-Aldrich, Israel) diluted 1:500. Cultures were centrifuged and supernatants were passed through an amylose column (NEB, New England Biolabs, Massachusetts, USA) previously equilibrated with activity buffer (100 mM Tris pH 8.0, 100 mM NaCl, and 100 μM MnCl₂). Protein was eluted with column buffer supplemented with 10 mM maltose, and loaded on a size exclusion chromatography (SEC) column, HiLoad® 16/600 Superdex® 75 pg column (GE Healthcare, Chicago, Illinois, USA), adapted for the AKTA fast protein liquid chromatography (FPLC) system and equilibrated with filtered column buffer. The purity of the fusion enzymes was established by 12% SDS-PAGE, and samples were stored at 4° C.

The codon optimized sequence of putative lactonase from P. expansum (named PELa) was ordered from GenScript (New Jersey, USA) cloned into an expression vector, pMAL-c4X, at its EcoRI and PstI sites. pMAL-c4X-PELa vector was used for lactonase expression as a fusion protein with MBP. Recombinant expression was performed in E. coli-BL21 (DE3) cells containing pGro7 plasmid (TAKARA, Ship, Japan), for co-expression with GroEL/ES as a chaperon, as described in Zhang et al., 2019. For this, pMal-PELa plasmid was transformed into E. coli-BL21 (DE3) cells, containing the pGro7 plasmid and plated on LB agar with 100 μg/mL ampicillin and 34 μg/mL chloramphenicol. These overnight cultures were used to inoculate (at 1:100 dilution) a fresh LB with 100 μg/mL ampicillin, 34 μg/mL chloramphenicol and 100 μM ZnCl₂, and 0.05% (w/v) arabinose, to induce GroEL/ES expression. Cells were grown at 30° C. with shaking to reach an OD₆₀₀ =0.6-0.8, then final concentration of 0.4 mM IPTG was added to induce overexpression. Protein purification was performed as described for PPH-G55V.

Enzyme kinetics analysis. The activity of PPH-G55V (0.3 μM) with patulin was analyzed using UV detection of patulin (Roach et al., 2002). For this 0.1 mM patulin in activity buffer: 100 mM Tris pH 7.5, 100 mM NaCl, 100 μM MnCl₂ was used for an absorbance scan, from 240-310 nm, at 24° C. (BioTeK (Winooski, Vermont, USA), optical length of ˜0.5 cm). The absorbance of patulin in activity buffer was measured at 278 nm in UV 96-well plates, with and without purified enzymes. Activity was monitored in a microtiter plate reader. Patulin's (in activity buffer) extension coefficient was calculated from the preformed calibration curve using patulin in increasing concentrations (0-0.4 mM). PPH-G55V activity was tested with different patulin concentrations (ranging from 0 to 0.3 mM). Reactions were performed at the same concentration of organic solvent, regardless of substrate concentration. Vo-enzyme initial rates were corrected for the background rate of patulin spontaneous hydrolysis in the absence of the enzyme. Kinetic parameters were obtained by GraphPad software as fitting initial rates directly to the Michaelis-Menten equation V₀=k_{cat}[E]₀[S]₀/([S]₀+K_{M}) (Wongsuk et al., 2016). Error ranges relate to the standard deviation of the data obtained from at least two independent measurements.

Addition of purified lactonase to P. expansum liquid culture. Purified lactonase at a final concentration of 2 μM was added to a 3 mL P. expansum culture in PDB medium containing 2.5×10³ spores, grown at 25° C., 150 rpm. After 3 days, mycelium growth was visually evaluated, and mycelium fraction was weighted following centrifugation for 10 min at 10,000 rpm (fresh weight). Mycelia treated with the enzyme's activity buffer was used as a control. Each treatment was consisted of 3 biological repeats.

Effect of purified lactonase on spores' germination and colony growth. P. expansum conidia were harvested from 5-day-old PDA plates. Conidia harvesting was performed by spreading 5 mL of 0.01% (v/v) Tween 20 (Sigma-Aldrich, Copenhagen, Denmark) in sterile ddH₂O (sterile double distilled water). Purified enzyme (2 μM) was added to 1 mL sterile water containing 2.5×10³ spores and incubated with shaking at 300 rpm at 25° C. Spore germination was observed microscopically (Micros Lotus MCX51, Gewerbezone, Austria) at a magnification of 40×; every 60 mM, 20 μL from the solution was examined with a hemocytometer (Assistant, Germany)

To test colony growth, P. expansum spores were collected from a colony that grew for 5 days. Spores (2 μL from 10⁶/mL solution in 1 mL of PDB medium) were incubated with shaking at 300 rpm in the presence of 2 μM PPH-G55V for 30 mM at 25° C. After incubation, a 10 μL aliquot of treated spores was spread on PDA plates and placed in the dark at room temperature for 48 h to test colony regeneration and development.

Pathogenicity assay of P. expansum in apples. P. expansum spores (10 μL of 2.5×10³) were incubated at 25° C. during 30 mM with 2 μM purified enzyme in 1 mL final volume (with sterile ddH₂ O) previously to inoculation in fruits. Freshly harvested apples cv. “Golden Delicious” were surface-cleaned with 70% ethanol and wound-inoculated by puncturing 2 mm deep with a sterile needle. Conidial suspension (10 μL) and the enzyme were placed on the wound and incubated at 24° C. Disease colonization was monitored daily for disease symptoms and lesion diameter. In each treatment, five different apples were inoculated 3 times per apple at the equatorial axis (5×3=15 replications). Spores incubated in enzyme buffer (100 mM Tris-HCl pH 8.0, 100 mM M NaCl, and 100 μM MnCl₂) without the enzyme were used as a control. To assess the effect of enzyme application time on disease development, apples were also sprayed with the purified lactonase, 30 min prior or post inoculation with spore suspensions on apples surface, and enzyme buffer was used as a control.

RNA isolation and quantitative real-time PCR (qPCR). RNA was extracted from grinded mycelia or from the leading edge of the decayed infected apple tissue, as previously described (Barad et al., 2014). RNA extraction was performed with a fungal total RNA purification kit (Norgen, Canada) according to the manufacturer's protocol. cDNA was then synthesized. Using the Verso cDNA Synthesis Kit (ThermoFisher Scientific, Waltham, Massachusetts, USA). qPCR was performed using the LightCycler Instrument II (Roche, Basel, Switzerland) in 384-well plates. PCR amplification was performed with 1 ng/μL cDNA template in 4 μL of a reaction mixture (LightCycler 480 SYBR Green I Master, Roche) containing 250 nM primers final concentration.

For qPCR analysis, the amplification program included one cycle of pre-incubation at 95° C. for 5 min, followed by 45 cycles of 95° C. for 10 s, 60° C. for 20 s, and 72° C. for 20 s followed by a melting curve analysis cycle of 95° C. for 5 s and 65° C. for 1 min. Relative quantification of all samples was normalized to 28 s expression levels and calculated using the ΔCt model (Yuan et al., 2006). The ACT value was determined by subtracting the CT results for the target gene from those for the endogenous 28 s control gene. As described by ΔCt target=Ct (reference gene)—Ct (target gene). Primer efficiency was established using serial dilutions of pooled cDNA and found to be equal to 1.98 (amplification factor per cycle). Efficiency was presumed to be the same for all samples. Therefore, the calculated expression ratio was: ratio=1.98^{ΔCt}. Each experiment was performed in three different biological replicates. For each biological sample the qPCR ran was conducted in four technical repetitions.

Sequence identification, alignment of putative lactonase, and structure modeling. Homologs were identified using the sequence of AiiA (WP_000216581.1), an AHL lactonase from B. thuringiensis previously characterized (Cui et al., 2021). The search was performed with the protein-alignment BLAST (blastp) function in the NCBI nonredundant protein sequence database (nr), and included several available genomes of bacterial spices such as Bacillus megaterium (basionym: Priestia megaterium) and fungal species such as P. expansum, Aspergillus clavatus, Penicillium digitatum, Pseudogymnoascus verrucosus, and Fonsecaea pedrosoi. The sequences of identified putative homologs and previously characterized MBL superfamily AHL lactonase (ranging from 59% to 78% identity between homologs), were aligned in MEGA X software (Kumar et al., 2018b). An alignment picture was created with the freely available software Jalview (Waterhouse et al., 2009).

A sequence with 29.25% identity, 77% coverage, and E-value of 2×10⁻²¹ was found in the genome of P. expansum (XP_016600436.1) annotated as hypothetical protein PEX2_072460. The putative enzyme was named here PELa, for P. expansum Lactonase. To further validate that the newly identified fungal enzyme is a lactonase, structural comparison with solved structure of bacterial AHL lactonase was performed. For this, a 3D structural model was generated by submitting XP_016600436.1 amino acid sequence to an online sever SWISS-MODEL (https://swissmodel.expasy.org/, accessed on 29.08.2020). SWISS-MODEL is an automated software that calculates structural models based of known solved structures used as templates, and sequence-structure alignments (Kumar et al., 2018b). Specifically, the solved structure of the AHL lactonase from Alicyclobacillus acidoterrestris, pdb (Protein Data Bank) number 6cgy was found as best hit by the server for modeling, and therefore it was used as a template for structural modeling of PELa. Next, the resulting structural model of PELa from P. expansum was aligned with the structure of AHL lactonase from Alicyclobacillus acidoterrestris (pdb 36cgy), using PyMOL Molecular Graphics System, Version 1.2r3pre, Schrödinger, LLC (New York, NY, USA).

Putative lactonase from P. expansum characterization. The codon-optimized sequence of putative lactonase from P. expansum (named PELa) was ordered synthetically from GenScript (New Jersey, USA) cloned into an expression vector, pMAL-c4X at its EcoRI and PstI sites. The pMAL-c4X vector was used for expression as a fusion protein with maltose binding protein (MBP), and protein was expressed and purified as described above. Purified PELa (0.6 μM) was incubated with 0.3 mM patulin at various temperatures to determine the optimal temperature for hydrolytic activity. Samples collected at time 0 and after 2 mM, were spectrophotometrically analyzed at 278 nm. The control sample (activity buffer; 100 mM Tris-HCl pH 7.5, 100 mM M NaCl, and 100 μM ZnCl₂) was incubated under the same conditions, and values were subtracted from each corresponding test sample containing the enzyme. Readings of pre-incubation samples were subtracted from the reading of post-incubation samples. A total of 100% activity was defined as the activity at 25° C. Each treatment was tested in triplicates. To test the optimal pH for activity, μM of purified PELa was diluted in activity buffer adjusted to pH values ranging between 3.5 and 11 (100 mM acetate buffer for pH 4.5-5.5, phosphate buffer for pH 5.5-8.0, Tris buffer for pH 8.0-9.0). Enzyme activity was measured at 25° C. for 15 mM (at higher temperatures, high spontaneous hydrolysis was observed), by adding 0.3 mM patulin, in the same buffer for each pH value. The spontaneous hydrolysis of patulin in enzyme-free activity buffer at each pH was subtracted from the hydrolysis measured in each corresponding test sample.

### Results

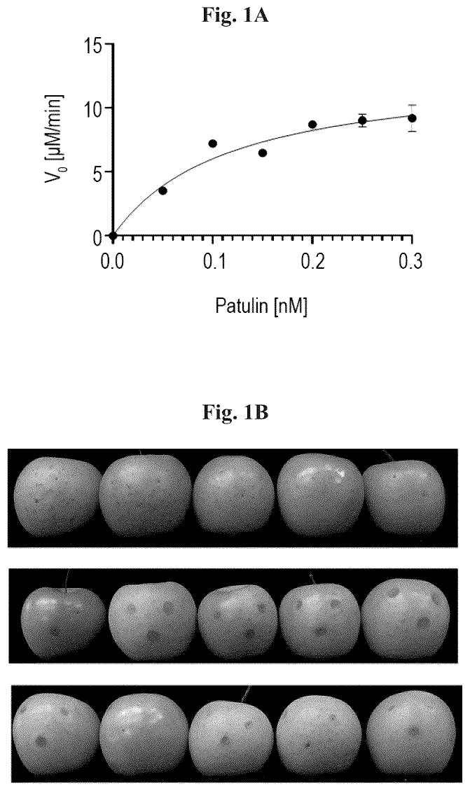

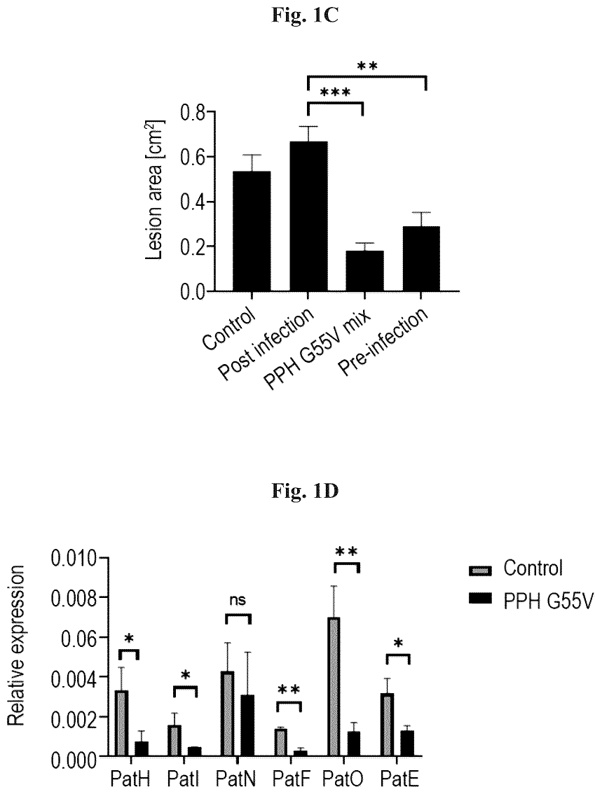

Bacterial quorum-quenching lactonase degrades patulin, inhibits apples infection, and modulates gene expression in P. expansum during infection. Following the UV absorbance of lactone-based mycotoxin patulin from P. expansum, at 278 nm, with extension coefficient of 8000 OD/M, (data not shown), enabled the detection of enzymatic activity of recombinant expressed and purified PPH-G55V with patulin (FIG. 1A). The bacterial enzyme exhibited considerably high catalytic efficiency, with a k_{cat} value of 0.724±0.077 s⁻¹ and K_{M} value of 116±33.98 μM. The calculated specific activity (k_{cat}/K_{M}) showed a value of 6.24×10³ s⁻¹ M⁻¹, which is one order of magnitude lower than its activity with bacterial QS molecules AHLs (Zhang et al., 2019).

Colonization pattern of P. expansum spores mixed with purified PPH-G55V before inoculation induced a 65% (*** p<0.0005) reduction of the lesion area in infected apples after three days (FIGS. 1B-D). Pre-inoculation spray of 2 μM PPH-G55V reduced the lesion area by 46% (** p<0.0047), while post inoculation treatment of the fruit with 2 μM PPH-G55V spray showed no effect on lesion development (FIG. 1E).

Analysis of the effect of the fungal enzyme mix before inoculation on the gene expression in the biosynthetic cluster of patulin during fungal colonization showed a relative inhibition ranging between 28% to 82% (FIG. 1F). Relative expression of PatH (encoding m-cresol methyl hydroxylase), Patl (encoding m-hydroxybenzyl alcohol hydroxylase), PatF (encoding neopatulin synthase), PatO (encoding putative isoamyl alcohol oxidase), and PatE (encoding glucose-methanol-choline), were significantly downregulated by the following percentages compared with the control without enzyme: 78%, 71%, 28%, 82%, and 58%, respectively (p<0.05 *, <0.003 **).

Bacterial lactonase modulates fungal growth of P. expansum and gene expression in culture. The addition of purified PPH-G55V to P. expansum spores grown on PDA solid media did not show any significant change in germination or colony development (data not shown). Growth of P. expansum spores performed in PDB liquid medium in the presence of the purified enzyme PPH-G55V showed a different pattern of hyphal morphology after three days of growth (FIG. 2A, right tube). Microscopic observations indicated thinner cell walls in hyphae grown with PPH-G55V than the hyphae from the fungal culture without the enzyme (FIG. 2B). The fresh weight of the fungal mycelium grown with the enzyme showed a ten-fold reduction compared with untreated mycelia (FIG. 2C). Sampling the treated mycelia and plating on fresh PDA plates showed apparent differences in the number of new colonies developed from the enzyme-treated mycelia. While hundreds of new small colonies developed from enzyme-treated mycelia, only about 30 colonies developed from control mycelial suspension (data not shown), suggesting an effect of the enzyme on mycelia.

Next, we tested the expression of several previously shown genes involved in fungal cell wall biosynthesis and morphogenesis. Such genes were identified in P. chrysogenum and P. expansum. In P. chrysogenum the genes are Bgt1 (Pc15g01030), accession number XP_014536515.1 and Gel1 (Pc13g08730), accession number XP_014538414.1, encoding for β(1-3) glucanosyltransferases (Jami et al., 2010). These enzymes play a role in the biosynthesis of fungal cell wall by elongating and remodeling β-1,3 glucan. In a recent predicted secretome analysis of P. expansum in fruit apple interactions, PEX2_048400, coding for 13(1-3)-transglycosylase was highly expressed (Levin et al., 2019). Therefore, based on XP_014536515.1 and XP_014538414 1 amino acid sequences, homologs were identified in the P. expansum genome (XP_016597554.1 and XP_016594412.1 with 89% and 94%, respectively), using NCBI protein blast. Primers as disclosed in Barad et al., 2016 were used. Next, qRT-PCR analysis indicated that both genes encoding homologs to Gel1 and Bgt1 were downregulated significantly by 37% and 48%, respectively, in enzyme-treated mycelia (* p=0.0267, * p=0.0435; see FIG. 2D), suggesting a possible effect of the enzyme on the cell wall biosynthesis of the P. expansum hyphae.

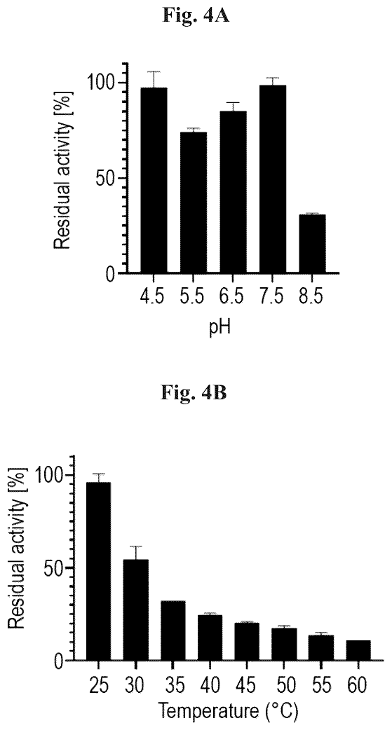

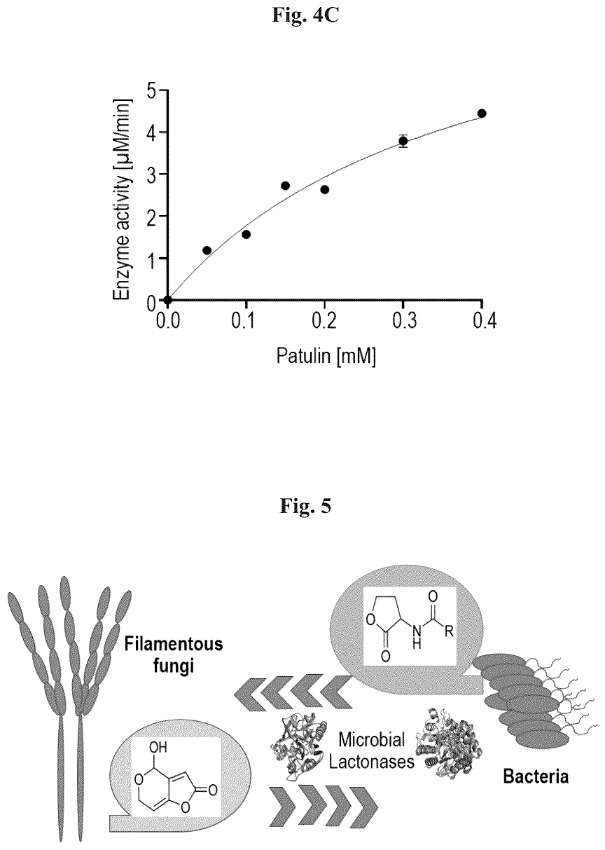

Identification of putative lactonases in fungal species and verification of activity with patulin for the homolog from P. expansum. As mentioned, one of the hypotheses regarding the role of bacterial AHL lactonases is that they self-regulate QS signaling within the same species, supported by the evidence that AHL-producing bacterial strains can also degrade them. We hypothesized that similarly, fungal-secreting patulin might encodes for a lactonase that degrade patulin for self-regulation or mycotoxin recycling. BLAST analysis using the amino acid sequence of bacterial AHL lactonase, PPH, as the query sequence; did not yield any homologs with above 28% identity in the NCBI-available genomes of P. expansum. However, a homolog was identified in P. expansum 072460, when the sequence of AiiA from Bacillus thuringiensis (WP_000216581.1) belonging to the MBL superfamily (Bebrone, 2007) was used as the query sequence. The homolog shared 29% identity and 76% coverage with an E value of 9×10⁻¹⁷. Similarly, homologs were identified in other 32 fungal species sharing 60-80% identity between them (data not shown). FIG. 3A presents the sequence alignment of AiiA from Bacillus thuringiensis and fungal species such as P. expansum, Aspergillus clavatus, Penicillium digitaturn. Pseudogymnoascus verrucosus, Fonsecaea pedrosoi, and Lindgoinyces ingoldianu. The alignment indicates that the newly identified fungal proteins are putative lactonases as they all possess a signature sequence, the HxHxDH˜H˜D˜H motif, common to lactonases in the MBL superfamily (Bebrone, 2007). A 3D homology model was predicted based on the amino acid sequences of the homolog from P. expansum using the solved structure of an AHL lactonase from Alicyclobacillus acidoterrestris (PDB 36cgy) as template. As shown in FIG. 3B, the structural overlay of the metal ion-coordinating residues in the active site of AHL lactonase from Alicyclobacillus acidoterrestris and the homology model of P. expansum, indicates that the two proteins share the same fold and bear a similar active site. Therefore, the synthetic gene of the homolog from P. expansum dubbed here PELa was cloned into an expression vector, recombinant expressed, and purified. The newly identified enzyme maintained its activity between pH values of 4.5-7.4, and its highest activity was detected at 25° C. (FIGS. 4A-4B). Michaelis-Menten analysis of the activity with patulin (FIG. 4C) showed a kcat value of 0.235±0.002 s⁻¹, KM value of 376.7±112 μM, and the calculated specific activity k_{cat}/K_{M} value of 6.24×10² s⁻¹ M⁻¹, an order of magnitude lower activity than that observed with PPH-G55V. These results indicate that P. expansum lactonase might be involved in self-regulation by patulin recycling. Towards the understanding of the involvement of lactonase activity with an ecological relevant bacteria in degrading fungal mycotoxins in the apple microbiome, the sequence of AiiA from Bacillus thuringiensis (WP_000216581.1) was used similarly to identify putative lactonases from bacteria that reside in apple trees, such as in Bacillus megaterium (basionym: Priestia megaterium). B. megaterium was identified in bark samples from apple and pear orchards and was suggested to be antagonistic to the AHL-producing pathogen Erwinia amylovora (E. amylovora) (Jock et al., 2002). Indeed, a homolog sharing 96% identity was identified, with the accession number ACX55098.1 annotated as AHL lactonase by NCBI.

### Discussion

The apple microbiome is comprehensively studied, and recent studies have shown that different apple fruit tissues (calyx-end, stem-end, peel, and mesocarp) harbor distinctly different fungal and bacterial communities that vary in diversity and abundance (Abdelfattah et al., 2021). However, few studies have focused on understanding the molecular mechanisms involving the interactions between epiphytic microbial (both bacterial and fungal) populations (Abdelfattah et al., 2021). One of the questions is related to the understanding of specific interactions, including metabolites and enzymes. This can increase the knowledge of using microbial antagonists as an alternative to synthetic chemicals in managing apples' postharvest bacterial and fungal pathogens.

Recently, an efficient AHL lactonase was identified and characterized in phytopathogen Erwinia amylovora (Jock et al., 2002). Furthermore, adding this purified enzyme to Erwinia amylovora cultures resulted in a lower relative expression level of bacterial QS-regulated genes. However, the ability of such bacterial lactonase to degrade lactone-based fungal mycotoxins was not explored, nor was the effect on fungal cultures, gene expression, and virulence. Here, we used a highly active and stable AHL lactonase (PPH-G55V) (Gurevich et al., 2021) to test these effects. Our results suggest a new function for bacterial AHL lactonases, with a hydrolytic mechanism, thus far known to degrade mainly bacterial AHLs, QS signaling molecules and act as quorum-quenching enzymes that reduce virulence plant pathogens. Patulin is a lactone-based fungal mycotoxin, shown to be related to pathogenicity affecting mycelium growth, and linked to host-pathogen-microbe interaction. We tested patulin degradation with the bacterial AHL lactonase. To test the bacterial enzyme effect on fungi culture and during apple infection, we recombinant expressed and purified the stabilized PPH-G55V. Present findings indicate that PPH-G55V could degrade patulin with a k_{cat}/K_{M} value of 6.21×10³ s⁻¹ M⁻¹, one to two orders of magnitude lower activity than its high efficiency with the bacterial AHLs (Wongsuk et al., 2016). At the same time, the capability of the bacterial lactonase to reduce pathogenicity in planta confirmed patulin suggested role as a factor contributing to pathogenicity of P. expansum, and the ability of this lactonase to reduce infection in apples.

Furthermore, the bacterial AHL lactonase added to fungal cultures inhibited the relative expression level of genes involved in patulin biosynthetic cluster during apple tissue colonization by P. expansum. The inhibited genes included PatH (m-cresol methyl hydroxylase), PatI (m-Hydroxybenzyl alcohol hydroxylase), PatO (putative isoamyl alcohol oxidase), and PatE (glucose-methanol-choline). The inhibition ranged from 28 up to 82%. Interestingly, the last precursors in patulin synthesis such as neopatulin and ascladiol possess lactone rings (Madsen et al., 2020), and the gene expression of their corresponding enzymes was significantly inhibited. This indicates that the bacterial lactonase may not only degrade patulin but also affect its biosynthetic pathways at the gene expression level, in a mechanism that is yet to be discovered. Recently, the bacterial Methylobacterium orvzae co-cultured with P. expansum, showed an inhibition of P. expansum, on patulin production and on the transcriptional level of the gene coding for isoepoxydon dehydrogenase (Afonso et al., 2021).

The bacterial enzyme PPH-G55V also showed a significant effect on the morphology of P. expansum growth. While the addition of bacterial enzyme PPH-G55V did not inhibit fungal growth in solid media, and no effect seen on germination, it did affect fungal development and mycelia production when added to mycelia in liquid media. It further reduced the expression of Gel1 and Bgt1 homologs, coding for enzymes known to be involved in fungal cell wall development to 66% and 52%, respectively. Interestingly, the regeneration of colonies from enzyme-treated mycelia in liquid culture resulted in a multi-development of colonies compared with the control. These results suggest that the lactonase activity induces a morphological change in liquid growth related to cell wall development. Therefore, we surmise a link between the reduction in P. expansum apple colonization caused by the addition of bacterial lactonase to fungal cultures, patulin metabolism and the changes in morphology via the downregulation of the biosynthetic cluster of patulin and cell wall development-related genes. These results also suggest that AHL lactonases may interfere with inter-kingdom communication between fungal and bacterial communities via their ability to degrade lactone-based mycotoxins (FIG. 5). As there are growing numbers of studies indicating that fungal mycotoxins play an essential role in inhibiting bacterial communications such as QS (Cui et al., 2021), the presented results suggest that bacterial AHL lactonase may disturb fungal communication signals in the apple microbial environment, acting in an antagonistic mechanism. Although M. tuberculosis (PPH bacterial origin) is not an epiphytic apple bacterium, we have previously identified and characterized a lactonase in an apple bacterial pathogen, E. amylovora, as a quorum-quenching lactonase (Gurevich et al., 2021). This bacterium is part of the epiphytic community in apples and pears trees; furthermore, we identified herein a putative lactonase sequence in Bacillus megaterium. Future work should test the effect of lactonases naturally expressed in bacteria co-cultured with P. expansum.

Since some bacteria that secret AHLs also express AHL lactonases, we surmised that the fungi would have patulin degrading lactonase activity. Indeed, putative lactonases homologs from the MBL fold were identified in various fungal species, and the activity of the recombinant expressed and purified homolog from P. expansum was verified with patulin, but with an order of magnitude lower activity than the activity measured with the bacterial lactonase.

While postharvest biocontrol products using microbial antagonists, especially yeasts, have been isolated from epiphytic communities of the fruits and commercialized, wide use is limited due to problems with efficacy and regulatory hurdles. A greater understanding of the fruit microbiome is needed to elucidate the factors involved in biocontrol systems. This would facilitate improved strategies that rely on antagonistic microorganisms or enzymes for managing postharvest diseases of fruit crops (Abdelfattah et al., 2021). The results presented here suggest that bacterial lactonases may be one mechanism used by antagonist species, and purified and stable enzymes may serve as a better strategy than using antagonistic bacteria, reliving microbial competition. Specifically, PPH-G55V can be further developed as pretreatment to reduce fungal damaged apples, by applying the enzyme on apples before storage. Another possible application is as a treatment for high patulin residual concentration in apple products.

### Conclusions

Bacterial quorum-quenching lactonases, thought to evolve towards the degradation of bacterial AHL molecules, are widely conserved in various bacterial species and have a variable substrate range. Here, the ability to degrade patulin by one such bacterial enzyme from the PTE-like lactonases family was described. Patulin is a lactone-based fungal mycotoxin. This lactonase activity appears to be correlated with inhibiting fungal colonization due to interfering with patulin concentration and synthesis, and cell wall morphology. The lactonase inhibitory effect is supported by reducing relative gene expression upon its addition to P. expansum cultures. Understanding the impact of patulin on beneficial or harmful microorganisms that reside within the microbiome and its enzymatic degradation can identify new antimicrobial methods to reduce fruit decay and decrease mycotoxin contamination.

Moreover, the degradation of patulin by bacterial lactonase presents a new method to study the interaction between bacteria and fungi communities. Patulin hydrolyzing activity by epiphytic bacteria can be referred to as part of inter-kingdom communication between fungi and bacteria. On the other hand, fungal lactonases might play a role in fungal self-regulation of patulin synthesis. Present results also suggest a potential application of quorum-quenching lactonases with patulin-degrading activity as a new approach for disease control of postharvest infection by P. expansum and other postharvest pathogens producing lactone mycotoxins.

Study 2. Recombinant EaAiiA lactonase degrades patulin and inhibits P. expansum colonization

Erwinia amylovora is a Gram-negative bacterium encoding an AHL lactonase homolog (EaAiiA) in its genome. Previously, we described the identification of a putative AHL lactonase homolog (EaAiiA), based on a structural modeling, as belonging to the metallo-β-lactamase (MBL) fold, and verified its AHL lactonase activity (Ya'ar Bar et al., 2021). In this Study we show that EaAiiA has similar rate of catalytic activity using the lactone patulin, with a k_{cat} value of 5.426±1.5 s⁻¹ and an estimated K M value of 764.2±284.4 μM, (FIG. 6A). The calculated specific activity (k_{cat}/K_{M}) showed a value of 7.10×10³ s⁻¹ M⁻¹, similar to the k_{cat}/K_{M} value of the previously characterized bacterial lactonase (PPH-G55V). When P. expansum spores were incubated in the presence of 2 μM EaAiiA lactonase prior to apple inoculation, the colonized area declined by 85% by the third day after inoculation (FIG. 6B). Inactivation of EaAiiA lactonase by exposure of the enzyme to 90° C. following mixture of the enzyme with 0.5 mM EDTA, prior to incubation with P. expansum spores, fully reduced the inhibition of fungal colonization of the pathogen (FIG. 6B). These results suggest that the lactonase has a dual effect on patulin catabolism which also contributes to the reduced pathogenicity of P. expansum on apples.

### Materials and Methods

Recombinant expression and purification of E. amylovora EaAiiA lactonase. E. amylovora EaAiiA was previously identified and characterized as an AHL lactonase (Ya'ar Bar et al., 2021). Its gene was cloned into pMAL-c2x vector for expression as a fusion protein with maltose binding protein (MBP). Recombinant expression and purification were performed as follow: pMAL-c2x-eaaiia was transformed to E. coli-BL21 (DE3) cells, plated on LB agar with 100 μg/ml ampicillin, and incubated at 37° C. for overnight. Next, a single colony was inoculated in 10 ml LB medium with 100 μg/mL ampicillin and 0.1 mM ZnCl₂, as this enzyme requires two metal ions in its active site for activity. Cultures were grown at 37° C., 170 rpm. Following overnight growth, cultures were inoculated (1:10) in 1.5 L fresh LB medium with 100 μg/mL ampicillin and 0.1 mM ZnCl₂, for ˜5 hours at 30° C., 170 rpm. When the culture reached OD₆₀₀=0.6-0.8, 0.4 mM IPTG (isopropyl β-d-1-thiogalactopyranoside) was added for protein expression and flowed by incubation overnight in the same conditions. The next day, cells were harvested by centrifugation, and the pellet was suspended in lysis buffer containing 100 mM Tris-HCl pH 8.0, 100 mM NaCl, 100 μM ZnCl₂ and protease inhibitor cocktail (Sigma-Aldrich, Israel) diluted 1:500 and subjected to sonication. Next, following centrifugation, the supernatant fraction was passed through an amylose column (NEB, New England Biolabs, Ipswich MA, USA) adapted for the ÄKTA pure™ chromatography system for protein purification. Column was equilibrated with filtered column buffer (100 mM Tris pH 8.0, 100 mM NaCl, and 100 μM ZnCl₂). Protein was eluted with column buffer supplemented with 10 mM maltose. The purity of the fusion enzymes was established by 12% SDS-PAGE, and samples were stored at 4° C.

Enzyme kinetics analysis with patulin. EaAiiA activity (0.05 μM) with patulin was analyzed as follows: EaAiiA activity was measured by detecting the reduction in the UV absorbance (detected at 278 nm) of patulin at different concentrations (0-0.4 mM) in the presence of enzyme at a final concentration of 0.05 μM, in an activity buffer containing 100 mM Tris pH 7.5, 100 mM NaCl, 100 μM ZnCl₂, in UV 96-well plates. An activity buffer was used as a negative control. The activity was monitored in a microtiter plate reader (BioTeK, Winooski, VT, USA). Reactions were performed as the concentration of organic solvent was lower than 1%. Vo initial rates were corrected for the patulin spontaneous hydrolysis background rate performed with an activity buffer. Kinetic parameters were obtained by GraphPad software as fitting initial rates directly to the Michaelis-Menten equation, standard deviations obtained from three independent measurements.

Microorganism and growth conditions. The plant pathogen Penicillium expansum Pe-21 was grown on PDA plates (Difco, Detroit, MI, USA) at room temperatures (22-24° C.) in the dark, or in liquid medium, PDB (Difco, Detroit, MI, USA) or LB (Formedium, Hunstanton, England) at 25° C. with shaking at 150 rpm. For Mycelial growth and fruit inoculation. One-week-old conidia, grown on PDA plates, were harvested by adding 5 mL of sterile distilled water with 0.01% (v/v) Tween 20 (Sigma-Aldrich, Copenhagen, Denmark), gently rubbing the fungal spores, and collecting to 1.5 mL tubes.

Pathogenicity assay in apples. P. expansum spores (50 spores/ml) were incubated in 1 ml LB medium/sterile water at 25° C. 300 rpm either alone or 2 μM EaAiiA, and apples were then inoculated and incubated in the same manner as described above. Enzyme incubated with 50 mM EDTA at 90° C. for 1 hour was used as a negative control (inactivated enzyme).

### REFERENCES

Abdelfattah, A.; Freilich, S.; Bartuv, R.; Zhimo, V. Y.; Kumar, A.; Biasi, A.; Salim, S.; Feygenberg, O.; Burchard, E.; Dardick, C.; et al. Global analysis of the apple fruit microbiome: Are all apples the same? Environ. Microbiol. 2021, 23(10), 6038-6055

Afonso, T. B.; Simões, L. C.; Lima, N. Effect of quorum sensing and quenching molecules on inter-kingdom biofilm formation by Penicillium expansum and bacteria. Biofouling 2020, 36, 965-976

Afonso, T. B.; Simões, L. C.; Lima, N. Methylobacterium oryzae influences isoepoxydon dehydrogenase gene expression and patulin production by penicillium expansum. Water 2021, 13, 1427

Aframian, N.; Eldar, A. A Bacterial tower of Babel: quorum-sensing signaling diversity and its evolution. Annu. Rev. Microbiol. 2020, 74, 587-606

Afriat, L.; Roodveldt, C.; Manco, G.; Tawfik, D. S. The latent promiscuity of newly identified microbial lactonases is linked to a recently diverged phosphotriesterase. Biochemistry 2006, 45, 13677-13686

Angeli, D.; Sare, A. R.; Jijakli, M. H.; Pertot, I.; Massart, S. Insights gained from metagenomic shotgun sequencing of apple fruit epiphytic microbiota. Postharvest Biol. Technol. 2019, 153, 96-106

Barad, S.; Horowitz, S. B.; Moskovitch, O.; Lichter, A.; Sherman, A.; Prusky, D. A penicillium expansum glucose oxidase-encoding gene, GOX2, is essential for gluconic acid production and acidification during colonization of deciduous fruit. Mol. Plant-Microbe Interact. 2012, 25, 779-788

Barad, S.; Horowitz, S.B.; Kobiler, I.; Sherman, A.; Prusky, D. Accumulation of the mycotoxin patulin in the presence of gluconic acid contributes to pathogenicity of penicillium expansum. Mol. Plant-Microbe Interact. 2014, 27, 66-77

Barad, S.; Espeso, E. A.; Sherman, A.; Prusky, D Ammonia activates pacC and patulin accumulation in an acidic environment during apple colonization by Penicillium expansum. Mol. Plant Pathol. 2016, 17(5), 727-740

Bebrone, C. Metallo-β-lactamases (classification, activity, genetic organization, structure, zinc coordination) and their superfamily Biochem. Pharmacol. 2007, 74, 1686-1701

Berg, G.; Köberl, M.; Rybakova, D.; Müller, H.; Grosch, R.; Smalla, K. Plant microbial diversity is suggested as the key to future biocontrol and health trends. FEMS Microbiol. Ecol. 2017, 93(5)

Cui, Z.; Huntley, R. B.; Zeng, Q.; Steven, B. Temporal and spatial dynamics in the apple flower microbiome in the presence of the phytopathogen Erwinia amylovora. ISME J. 2021, 15, 318-329

Fuqua, C.; Winans, S. C.; Greenberg, E. P. Census and consensus in bacterial ecosystems: the LuxR-Luxl family of quorum-sensing transcriptional regulators. Annu. Rev. Microbiol 1996, 727-751

Gurevich, D.; Dor, S.; Erov, M.; Dan, Y.; Moy, J. C.; Mairesse, O.; Dafny-Yelin, M.; Adler-Abramovich, L.; Afriat-Jurnou, L. Directed enzyme evolution and encapsulation in peptide nanospheres of quorum quenching lactonase as an antibacterial treatment against plant pathogen. ACS Appl. Mater. Interfaces 2021, 13, 2179-2188

Hadas, Y.; Goldberg, I.; Pines, O.; Prusky, D. Involvement of gluconic acid and glucose oxidase in the pathogenicity of Penicillium expansum in apples. Phytopathology 2007, 97, 384-390

Hornby, J. M.; Jensen, E. C.; Lisec, A. D.; Tasto, J. J.; Jahnke, B.; Shoemaker, R.; Dussault, P.; Nickerson, K. W. Quorum sensing in the dimorphic fungus Candida albicans is mediated by farnesol. Appl. Environ. Microbiol. 2001, 67, 2982-2992

Ianiri, G.; Idnurm, A.; Wright, S. A. I.; Duran-Patron, R.; Mannina, L.; Ferracane, R.; Ritieni, A.; Castoria, R. Searching for genes responsible for patulin degradation in a biocontrol yeast provides insight into the basis for resistance to this mycotoxin. Appl. Environ. Microbiol. 2013, 79, 3101-3115

Jami, M. S.; García-Estrada, C.; Barreiro, C.; Cuadrado, A. A.; Salehi-Najafabadi, Z.; Martin, J. F. The penicillium chrysogenum extracellular proteome. Conversion from a food-rotting strain to a versatile cell factory for white biotechnology. Mol. Cell. Proteom. 2010, 9, 2729-2744

Jock, S.; Lksch, B.; Mansvelt, L.; Geider, K. Characterization of Bacillus strains from apple and pear trees in South Africa antagonistic to Erwinia amylovora. FEMS Microbiol. Lett. 2002, 211, 247-252

Kumar, D.; Barad, S.; Chen, Y.; Luo, X.; Tannous, J.; Dubey, A.; Glam Matana, N.; Tian, S.; Li, B.; Keller, N.; et al. LaeA regulation of secondary metabolism modulates virulence in Penicillium expansum and is mediated by sucrose. Mol. Plant Pathol. 2017, 18, 1150-1163

Kumar, D.; Tannous, J.; Sionov, E.; Keller, N.; Prusky, D. Apple intrinsic factors modulating the global regulator, LaeA, the patulin gene cluster and patulin accumulation during fruit colonization by Penicillium expansum. Front. Plant Sci. 2018a, 9, 1094

Kumar, S.; Stecher, G.; Li, M.; Knyaz, C.; Tamura, K. MEGA X: Molecular evolutionary genetics analysis across computing platforms. Mol. Biol. Evol. 2018b, 35, 1547-1549

Levin, E.; Kishore, A.; Ballester, A.R.; Raphael, G.; Feigenberg, O.; Liu, Y.; Norelli, J.; Gonzalez-Candelas, L.; Wisniewski, M.; Droby, S. Identification of pathogenicity-related genes and the role of a subtilisin-related peptidase S8 (PePRT) in authophagy and virulence of Penicillium expansum on apples. Postharvest Biol. Technol. 2019, 149, 209-220

Lin, Y. H.; Xu, J. L.; Hu, J.; Wang, L. H.; Leong Ong, S.; Renton Leadbetter, J.; Zhang, L. H. Acyl-homoserine lactone acylase from Ralstonia strain XJ12B represents a novel and potent class of quorum-quenching enzymes. Mol. Microbiol. 2003, 47, 849-860

Liu, D.; Thomas, P. W.; Momb, J.; Hoang, Q. Q.; Petsko, G. A.; Ringe, D.; Fast, W. Structure and specificity of a quorum-quenching lactonase (AiiB) from Agrobacterium tumefaciens. Biochemistry 2007, 46, 11789-11799

Luciano-Rosario, D.; Keller, N. P.; Jurick, W. M. Penicillium expansum: Biology, omics, and management tools for a global postharvest pathogen causing blue mould of pome fruit. Mol. Plant Pathol. 2020, 21, 1391-1404

Madsen, A. M.; Frederiksen, M. W.; Jacobsen, M. H.; Tendal, K. Towards a risk evaluation of workers' exposure to handborne and airborne microbial species as exemplified with waste collection workers. Environ. Res. 2020, 183, 109177

Poonguzhali, S.; Madhaiyan, M.; Sa, T. Production of acyl-homoserine lactone quorum-sensing signals is wide-spread in gram-negative Methylobacterium. J. Microbiol. Biotechnol. 2007, 17, 226-233

Rasmussen, T. B.; Skindersoe, M. E.; Bjarnsholt, T.; Phipps, R. K.; Christensen, K. B.; Jensen, P. O.; Andersen, J. B.; Koch, B.; Larsen, T. O.; Hentzer, M.; et al. Identity and effects of quorum-sensing inhibitors produced by Penicillium species. Microbiology 2005, 151 (Pt 5), 1325-1340

Remy, B.; Plener, L.; Elias, M.; Daude, D.; Chabriere, E. Enzymes for disrupting bacterial communication, an alternative to antibiotics? Ann. Pharm. Fr. 2016, 74, 413-420

Remy, B.; Mion, S.; Plener, L.; Elias, M.; Chabrière, E.; Daudé, D. Interference in bacterial quorum sensing: A biopharmaceutical perspective. Front. Pharmacol. 2018, 9, 203

Roach, J. A. G.; Brause, A. R.; Eisele, T. A.; Rupp, H.S. HPLC detection of patulin in apple juice with GC/MS confirmation of patulin identity. Adv. Exp. Med. Biol. 2002, 504, 135-140

Rodrigues, C.F.; C̆ernáková, L. Farnesol and tyrosol: Secondary metabolites with a crucial quorum-sensing role in candida biofilm development. Genes 2020, 11, 444

Uroz, S.; Dessaux, Y.; Oger, P. Quorum sensing and quorum quenching: The Yin and Yang of bacterial communication. ChemBioChem 2009, 10, 205-216

Venkatesh, N.; Keller, N. P. Mycotoxins in conversation with bacteria and fungi. Front. Microbiol. 2019, 10, 1-10

Von Bodman, S. B.; Bauer, W. D.; Coplin, D. L. Quorum sensing in plant-pathogenic bacteria. Annu. Rev. Phytopathol. 2003, 41, 455-482

Walsh, T. J.; Groll, A.; Hiemenz, J.; Fleming, R.; Roilides, E.; Anaissie, E. Infections due to emerging and uncommon medically important fungal pathogens. Clin. Microbiol. Infect. 2004, (Suppl. Si), 48-66

Waterhouse, A. M.; Procter, J. B.; Martin, D. M. A.; Clamp, M.; Barton, G. J. Jalview Version 2-A multiple sequence alignment editor and analysis workbench. Bioinformatics 2009, 25, 1189-1191

Waters, C. M.; Bassler, B. L. Quorum sensing: cell-to-cell communication in bacteria. Annu. Rev. Cell Dev. Biol. 2005, 21, 319-346

Whitehead, S. R.; Wisniewski, M. E.; Droby, S.; Abdelfattah, A.; Freilich, S.; Mazzola, M. The Apple Microbiome: Structure, Function, and Manipulation for Improved Plant Health. In The Apple Genome, Compendium of Plant Genomes; Korban, S.S., Ed.; Springer: Cham, Switzerland, 2021

Wongsuk, T.; Pumeesat, P.; Luplertlop, N. Fungal quorum sensing molecules: Role in fungal morphogenesis and pathogenicity. J. Basic Microbiol. 2016, 56, 440-447

Ya'ar Bar, S.; Dor, S.; Erov, M.; Afriat-Jurnou, L., Identification and characterization of a new quorum-quenching N-acyl homoserine lactonase in the plant pathogen Erwinia amylovora. J. Agric. Food Chem., 2021, 69(20):5652-5662

Yuan, J. S.; Reed, A.; Chen, F.; Stewart, C.N. Statistical analysis of real-time PCR data. BMC Bioinform. 2006, 7, 1-12

Zhang, J. W.; Xuan, C. G.; Lu, C. H.; Guo, S.; Yu, J. F.; Asif, M.; Jiang, W. J.; Zhou, Z. G.; Luo, Z. Q.; Zhang, L. Q. AidB, a novel thermostable N-acylhomoserine lactonase from the bacterium Bosea sp. Appl. Environ. Microbiol. 2019, 85(24), e02065-19

What is claimed is:

1. A method for treating or preventing infection of a fungus in a plant or a part, organ or a propagation material thereof, or in a product made from said plant, part, organ or propagation material, said fungus secreting patulin, and said method comprising applying a lactonase or a functional fragment thereof on said plant, part, organ or propagation material; or to said product, wherein said lactonase is (i) an acyl-homoserine lactonase; or (ii) the wild-type putative parathion hydrolase from M. tuberclorosis (PPH; SEQ ID NO: 1), or a phosphotriesterase-like lactonase having at least 30% identity to said wild-type PPH, a TIM-barrel fold substantially identical to that of the wild-type PPH, and preserved catalytic residues in its active site.

2. The method of claim 1, wherein said phosphotriesterase-like lactonase is a mutated phosphotriesterase-like lactonase in which a glycine residue corresponding to G59 of SEQ ID NO:
1 is substituted by an amino acid residue selected from the group consisting of valine, alanine, leucine, and isoleucine, or a histidine residue corresponding to H172 of SEQ ID NO: 1 is substituted by an amino acid residue selected from the group consisting of tyrosine, phenylalanine and tryptophan.

3. The method of claim 2, wherein the glycine residue corresponding to G59 of SEQ ID NO: 1 is substituted by valine.

4. The method of claim 3, wherein said mutated phosphotriesterase-like lactonase comprises or essentially consists of the amino acid sequence as set forth in SEQ ID NO: 2.

5. The method of claim 2, wherein the histidine residue corresponding to H172 of SEQ ID NO: 1 is substituted by tyrosine.

6. The method of claim 5, wherein said mutated phosphotriesterase-like lactonase comprises or essentially consists of the amino acid sequence as set forth in SEQ ID NO: 3.

7. The method of claim 1, wherein said phosphotriesterase-like lactonase has an increased thermostability in comparison with thermostability of a non-mutated wild-type phosphotriesterase-like lactonase or substantially similar or higher lactonase catalytic activity provided with N-(3-oxo-hexanoyl)-homoserine lactone as a substrate in comparison with said non-mutated phosphotriesterase-like lactonase.

8. The method of claim 7, wherein said increased thermostability expressed as T₅₀ is about to about 80° C.; and/or said mutated phosphotriesterase-like lactonase has an extended shelf-life as compared with said non-mutated phosphotriesterase-like lactonase.

9. The method of claim 2, wherein said phosphotriesterase-like lactonase is a mutated phosphotriesterase-like lactonase in which the glycine residue corresponding to G59 of SEQ ID NO: 1 is substituted by valine or the histidine residue corresponding to H172 of SEQ ID NO: 1 is substituted by tyrosine; and said mutated phosphotriesterase-like lactonase has an increased thermostability in comparison with thermostability of a non-mutated wild-type phosphotriesterase-like lactonase or substantially similar or higher lactonase catalytic activity provided with N-(3-oxo-hexanoyl)-homoserine lactone as a substrate in comparison with said non-mutated phosphotriesterase-like lactonase. The method of claim 9, wherein said mutated phosphotriesterase-like lactonase comprises or essentially consists of the amino acid sequence as set forth in SEQ ID NO: 2 or SEQ ID NO: 3;
said increased thermostability expressed as T₅₀ is about 55° C. to about 80° C.; and/or said mutated phosphotriesterase-like lactonase has an extended shelf-life as compared with said non-mutated phosphotriesterase-like lactonase.

11. The method of claim 1, wherein said acyl-homoserine lactonase is the wild-type putative acyl-homoserine lactonase from E. amylovora (EaAiiA; SEQ ID NO: 7), or an acyl-homoserine lactonase having at least 30% identity to said wild-type EaAiiA, a MBL fold substantially identical to that of the wild-type EaAiiA, and preserved catalytic residues in its active site.

12. The method of claim 1, wherein said fungus is of a genus selected from the group consisting of Penicillium, Aspergillus and Byssochlamys.

13. The method of claim 12, wherein said fungus is P. expansum.

14. The method of claim 1, wherein said plant is selected from the group consisting of apple tree, cherry tree, blueberry shrub, plum tree, banana tree, strawberry bush, grape vine, barley grain, wheat grain, and corn grain; said organ is a fruit of said plant; and said product is selected from the group consisting of sauce, juice, jam, or an alcoholic beverage, made from said fruit, and barley, wheat or corn flour. A method for reducing the concentration of patulin in a plant or a part, organ or a propagation material thereof; in a product made from said plant, part, organ or propagation material; or in a non-plant food product, said method comprising applying a lactonase or a functional fragment thereof on said plant, part, organ or propagation material; or to said product, wherein said lactonase is (i) an acyl-homoserine lactonase; or (ii) the wild-type putative parathion hydrolase from M. tuberclorosis (PPH; SEQ ID NO: 1), or a phosphotriesterase-like lactonase having at least 30% identity to said wild-type PPH, a TIM-barrel fold substantially identical to that of the PPH, and preserved catalytic residues in its active site.

16. The method of claim 15, wherein said phosphotriesterase-like lactonase is a mutated phosphotriesterase-like lactonase, in which a glycine residue corresponding to G59 of SEQ ID NO: 1 is substituted by an amino acid residue selected from the group consisting of valine, alanine, leucine, and isoleucine, or a histidine residue corresponding to H172 of SEQ ID NO: 1 is substituted by an amino acid residue selected from the group consisting of tyrosine, phenylalanine and tryptophan.

17. The method of claim 16, wherein the glycine residue corresponding to G59 of SEQ ID NO:
1 is substituted by valine.

18. The method of claim 17, wherein said mutated phosphotriesterase-like lactonase comprises or essentially consists of the amino acid sequence as set forth in SEQ ID NO: 2.

19. The method of claim 16, wherein the histidine residue corresponding to H172 of SEQ ID NO: 1 is substituted by tyrosine. The method of claim 19, wherein said mutated phosphotriesterase-like lactonase comprises or essentially consists of the amino acid sequence as set forth in SEQ ID NO: 3.

21. The method of claim 14, wherein said phosphotriesterase-like lactonase has an increased thermostability in comparison with thermostability of a non-mutated wild-type phosphotriesterase-like lactonase or substantially similar or higher lactonase catalytic activity provided with N-(3-oxo-hexanoyl)-homoserine lactone as a substrate in comparison with said non-mutated phosphotriesterase-like lactonase.

22. The method of claim 21, wherein said increased thermostability expressed as T₅₀ is about to about 80° C.; and/or said mutated phosphotriesterase-like lactonase has an extended shelf-life as compared with said non-mutated phosphotriesterase-like lactonase.

23. The method of claim 16, wherein said phosphotriesterase-like lactonase is a mutated phosphotriesterase-like lactonase in which the glycine residue corresponding to G59 of SEQ ID NO: 1 is substituted by valine or the histidine residue corresponding to H172 of SEQ ID NO: 1 is substituted by tyrosine; and said mutated phosphotriesterase-like lactonase has an increased thermostability in comparison with thermostability of a non-mutated wild-type phosphotriesterase-like lactonase or substantially similar or higher lactonase catalytic activity provided with N-(3-oxo-hexanoyl)-homoserine lactone as a substrate in comparison with said non-mutated phosphotriesterase-like lactonase.

24. The method of claim 23, wherein said mutated phosphotriesterase-like lactonase comprises or essentially consists of the amino acid sequence as set forth in SEQ ID NO: 2 or SEQ ID NO: 3;
said increased thermostability expressed as T₅₀ is about 55° C. to about 80° C.; and/or said mutated phosphotriesterase-like lactonase has an extended shelf-life as compared with said non-mutated phosphotriesterase-like lactonase.

25. The method of claim 15, wherein said acyl-homoserine lactonase is the wild-type putative acyl-homoserine lactonase from E. amylovora (EaAiiA; SEQ ID NO: 7), or an acyl-homoserine lactonase having at least 30% identity to said wild-type EaAiiA, a MBL fold substantially identical to that of the wild-type EaAiiA, and preserved catalytic residues in its active site.

26. The method of claim 15, wherein said plant is selected from the group consisting of apple tree, cherry tree, blueberry shrub, plum tree, banana tree, strawberry bush, grape vine, barley grain, wheat grain, and corn grain; said organ is a fruit of said plant; said plant product is a sauce, juice, jam, or an alcoholic beverage, made from said fruit; and said non-plant food product is shellfish.
